(19) United States
(12) Patent Application Publication
(10) Pub. No.: US 2023/0226109 A1
(43) Pub. Date: Jul. 20, 2023

(54) METHOD FOR DIFFERENTIATING INNATE LYMPHOID CELLS FOR IMMUNOTHERAPY

(71) Applicant: The Board of Trustees of the Leland Stanford Junior University, Stanford, CA (US)

(72) Inventors: John Sunwoo, Stanford, CA (US); Uriel Y. Moreno Nieves, Palo Alto, CA (US); Joshua Tay, Redwood City, CA (US); Saumyaa Saumyaa, Redwood City, CA (US); June H. Shin, Redwood City, CA (US); Nina Beryl Horowitz, Armonk, NY (US)

(21) Appl. No.: 17/766,019

(22) PCT Filed: Oct. 2, 2020
(86) PCT No.: PCT/US20/54055

§ 371 Date: Apr. 1, 2022

Related U.S. Application Data
(60) Provisional application No. 62/910,060, filed on Oct. 3, 2019.

(51) Int. Cl. A61K 35/17; C12N 5/0783; C07K 14/705; A61P 35/00

(52) U.S. Cl. CPC A61K 35/17; C12N 5/0646; C07K 14/7055; A61P 35/00; A61K 2039/5156

(57) ABSTRACT

Natural killer cells are differentiated to an intraepithelial innate lymphoid cells (ielLC1)-like cell, with an increase in cytotoxic activity. Specifically, the disclosure provides a method for differentiating mammalian natural killer cells to adapt an ielLC1-like phenotype, the method comprising: differentiating peripheral natural killer (NK) cells in the presence of IL-15 and epithelial cells or plate coatings that mimic features of epithelial cells, to generate CD49a+ CD103+ cells having features and phenotype of ielLC1s, with enhanced cytotoxic activity and expression of Th1 type cytokines.

### CROSS REFERENCE

This application claims priority to U.S. Provisional Application No. 62/910,060, filed Oct. 3, 2019 which is incorporated herein in its entirety for all purposes.

### GOVERNMENT SUPPORT

This invention was made with Government support under contract R01 CA158516 awarded by the National Institutes of Health. The Government has certain rights in the invention.

### BACKGROUND

The manipulation of cells, particularly immune cells, to differentiate, develop specialized functions and expand in numbers is of great clinical interest. In particular, developing immune cells that have activity against cancer cells has been of interest. While these approaches have been developed for antigen-specific T lymphocytes, there is also an opportunity for the use of lymphoid cells that lack antigen receptors. Recent developments have pointed to a diversity of innate immune lymphoid cells, including without limitation natural killer (NK) cells. These cells vary in their effector functions, tissue residence, cytokine responsiveness, and the like. The activity of these cells against target immune cells may be exploited in therapeutic modalities.

A critical challenge in cell-based therapies is differentiating cells for adoptive transfer to have desired behaviors, such as cytotoxicity, activation, expansion, etc. Methods are provided for this purpose.

### SUMMARY

Compositions and methods are provided for differentiating mammalian natural killer cells to adapt an intraepithelial innate lymphoid cells (ielLC1)-like phenotype. The differentiated cells have enhanced anti-tumor activity relative to the starting cell population. In some embodiments the cells are human cells. In some embodiments the cells are differentiated ex vivo. In other embodiments the cells are differentiated in vivo. The ex vivo differentiated cells are useful for adoptive transfer in, e.g. cancer treatment. A benefit of the use of the differentiated cells for cancer treatment is that they are not antigen specific, and have broader applicability to different tumor types. Without limitation, the increased cytotoxicity may be attributed to increased release of cytotoxic agents, enhanced release of cytokines, such as IFN-γ, better tissue residency and infiltration, and the potential to proliferate within tumors.

In the methods of differentiation, a population of peripheral NK cells are contacted with an effective dose of IL-15, optionally in combination with TGF-beta, and with epithelial cells or plate coatings that mimic epithelial cell attributes. The NK cells may be purified or otherwise enriched from a blood sample. In some embodiments the epithelial cells are carcinoma cells. In some embodiments the epithelial cells are squamous cell carcinoma cells. In some embodiments the epithelial cells are head and neck squamous cell carcinoma cells (HNSCC). The epithelial cells may be primary cells or cell lines. The epithelial cells can be irradiated prior to culture. The ratio of NK cells to target (cancer) cells may be from about 100:1, 50:1, 10:1, 5:1, 2:1, 1:1, 1:2, 1:5, 1:10, 1:50, 1:100, etc. Following a period of culture of from about 1, about 2, about 4, and usually not more than about 7 days, the cells thus differentiated can be isolated by selecting for ielLC1 markers, e.g. by selecting for cells that are positive for one or both of CD49a, and CD103. This method can be used in combination with methods for expansion of NK cells, which can be performed for a period of time in culture of from about 1, about 2, about 4, and usually not more than about 30 days. The methods are optionally combined with other methods of expanding NK cells.

In methods of use, an effective dose of differentiated ielLC1-like are administered to a mammal to provide for cytoxocity, e.g, against cancer cells, virus-infected cells, and the like. The cancer cells may be a hematologic cancer, or a solid tumor, including without limitation carcinomas. Administration may be systemic or intra-tumoral. The administration of cells may be combined with additional anti-cancer therapies, including checkpoint inhibitors, tumor-specific antibodies, as well as non-biologic therapies such as chemotherapy, radiation, etc.

The differentiated cell can be provided in a unit dose for therapy, and can be allogeneic, autologous, etc. with respect to an intended recipient.

phenotype or the ielLC1 phenotype. In some embodiments a method is provided for differentiating mammalian natural killer cells to adapt an intraepithelial innate lymphoid cells (ielLC1)-like phenotype, the method comprising differentiating peripheral natural killer (NK) cells in the presence of IL-15 and epithelial cells or plate coatings that mimic features of epithelial cells, to generate CD49a+ CD103+ cells having features and phenotype of ielLC1s, with enhanced cytotoxic activity and expression of Th1 type cytokines.

In some embodiments, following a period of culture of from about 1 to about 7 days, the cells thus differentiated are isolated by selecting for ielLC1 markers, including positive expression of one or both of CD49a and CD103. The NK cells may be human cells; and can be isolated or otherwise enriched from a human blood sample, differentiated from iPSC cells, or differentiated from NK cell precursors. The NK cells may be genetically modified to express a chimeric antigen receptor (CAR) construct, either prior to or subsequent to the differentiation step. The NK cells may be cryopreserved prior to differentiation.

It is shown herein that IL-15 promotes differentiation of peripheral NK into ielLC1-like cells, in vitro. IL-15 is also shown to promote. differentiation of peripheral NK into ielLC1-like cells, in vivo, Expression of CD49a and CD103 on gated Lin⁻CD45⁺CD56⁺ NK cells, after culture of NK with epithelial cells and IL15, for example at 10 ng/mL provides for increased percentage of CD49a³⁰ CD103⁺ on NK cells, after culture; and a functional response of in vitro differentiated ielLC1-like cells of increased anti-tumor effector function. Co-culture of peripheral NK cells with IL-15 and TGFβ enhances differentiation into ielLC1-like cells.

When the differentiating step is performed in vitro, the medium may comprise an effective dose of IL-15, optionally TGF-β, and either epithelial cells or a non-cellular matrix. Where epithelial cells are included, the cells can be irradiated prior to the culture step, e.g. where the epithelial cells are cancer cells, such as carcinoma cells, including without limitation head and neck squamous cell carcinoma cells (HNSCC). The ratio of NK cells to epithelial cells can be from about 100:1 to about 1:100. Where a non-cellular matrix is provided, the matrix may comprise plastic tissue culture plates coated with E-Cadherin in the absence of feeder layer cells. During the differentiation step, NKp44 may be activated in the differentiating step, where NKp44 ligand is shown to enhance ielLC1-like cell differentiation. During differentiation the aryl hydrocarbon receptor (AHR) activity can be modulated, where, for example, there was profound reduction of ielLC1-like differentiation with AHR inhibitor, and AHR activating ligands promoted the ielLC1-like differentiation. During the differentiation step, NR4A gene(s) may be inhibited to enhance cytotoxicity.

CAR-NK cells induced to differentiate into ielLC1-like cells have superior killing capacity. NK cells, transduced to express chimeric antigen receptor targeting CD19, were co-cultured with IL-15 and PCI-13 carcinoma cells. Killing against squamous cell carcinoma cells targeted by the CAR was shown to be superior to conventional CAR-NK cells.

The differentiated ielLC1-like cells can be administered treating a condition that benefits from enhanced cytotoxic activity and expression of Th1 type cytokines. In some embodiments the condition is cancer, e.g. hematologic cancer or a solid tumor. In some embodiments a solid tumor is a carcinoma, including without limitation HNSCC. In other embodiments the condition is an infection, e.g. a viral infection.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1: Human HNSCC contains several populations of innate lymphoid cells. (A) Workflow of the phenotypic and functional characterization of innate lymphoid cells from human blood and primary tumor tissue samples. In brief, blood and HNSCC tumor tissue samples were processed into single cell suspensions, which were stimulated or not, and then were either analyzed by conventional flow cytometry or were single-cell sorted for single-cell RNA sequencing analysis. (B-D) Single-cell RNA sequencing analysis of innate lymphoid cells from 8 different patients, (B) UMAP of peripheral and intra-tumoral innate lymphoid cell clusters, showing 8 distinct clusters. These include an ielLC1 cluster as well as an ielLC1-cycling cluster, showing the proliferation of ielLC1 cells within tumors. (C) Heatmap of marker genes for UMAP clusters, (D) Distribution of intra-tumoral innate lymphoid cell types across tumor tissue samples obtained from different patients.

FIG. 2: Differentially expressed marker genes across clusters. (A) LogFC plot comparing innate lymphoid cell clusters and their representative markers by gene expression. (B) Feature plots for marker genes. Note, NK-2 cells have high expression of NR4A1, NR4A2, and NR4A3. These genes are known to confer hyporesponsiveness. The NK-2 cells are the other terminal differentiation subset of NK cells, and NR4A expressing cells have reduced functional capacity in our functional studies.

FIG. 3: Flow cytometric analysis confirms heterogenicity of innate lymphoid cell populations in human HNSCC. (A-B) Single-cell suspensions of human HNSCC tumor specimens were stained for surface and intracellular markers. (A) Starting on gated Lin⁻CD56^{+/-}CD127^{+/-} as described in Supplemental FIG. 1, dot plots show the gating on ielLC1, ielLC1 cycling, ILC1, ILC3 and cNK cell populations by flow cytometry, (B) Histograms show expression of transcription factors Eomes, Tbet and RORgt on gated cNK, CD49a+, ILC1 and ILC3 populations.

FIG. 4: Intratumoral ILCs have distinct differentiation trajectories. (a) Pseudotime trajectories of cells that had primarily NK cell signatures, determined by CIBERSORTx deconvolution of the subset clusters, demonstrated two possible differentiation trajectories from peripheral NK cells: an end-state of either the NK-2 phenotype or the ielLC1 phenotype. (b) Expression of selected differentially-expressed genes along the NK-2 and ielLC1 pseudotime trajectories. Curves represent Loess-smoothed regression of the gene expression along each trajectory.

FIG. 5: IL-15 promotes differentiation of peripheral NK into ielLC1-like cells, in vitro. (A) Protocol for in vitro differentiation of peripheral NK cells into ielLC1-like cells. In brief, PCI13 cells were seeded into a culture plate, then peripheral NK cells were sorted and added to the culture (ratio 10NK:1PCI13) in the presence or not of IL15 (10 ng/mL). After 4 days of co-culture, NK cells were harvested for phenotypic and functional analysis. (B, LEFT) Dot plots show the expression of CD49a and CD103 on gated Lin⁻CD45⁺CD56⁺ NK cells, after culture of NK with PCI13 and IL15 (10 ng/mL) or control conditions. (MIDDLE) Graph shows cumulative results of the percentage of CD49a⁺ on NK cells, after culture of NK cells with PCI13 and IL15 (10 ng/mL) or control conditions. (RIGHT) Graph shows cumulative results of the percentage of CD49a⁺CD103⁺ on NK cells, after culture of NK cells with PCI13 and IL15 (10 ng/mL) or control conditions. (C-D) Functional response of in vitro differentiated ielLC1-like cells. (C, LEFT) Histogram shows production of IFNg by CD49a⁻ and CD49a⁺ cells differentiated with PCI13 only, in response to IL12+IL15+IL18, K562, PMA/lono or control condition (unstimulated). (C, RIGHT) Histogram shows production of IFNg by CD49a⁻ and CD49a⁺ cells differentiated with PCI13 and IL15 (10 ng/mL), in response to IL12+IL15+IL18, K562, PMA/lono or control condition (unstimulated). (D) After in vitro differentiation, CD49a⁻ and CD49a⁺ cells were sorted and mixed with PCI13 (10⁶ NK cells:5.10⁶ PCI13) and injected subcutaneously in the flank of NSG mice. Then, tumor volume was measured over time. This shows the importance of IL15 in the differentiation of cNK into ielLC1 in vitro. It also demonstrates the superior anti-tumor effector function of the CD49a+ ielLC1-like cells. (E) Sorted CD49a⁺ and CD49a⁻ NK cells were assessed for expression of NR4A1, NR4A2, and NR4A3 by qRT-PCR.

FIG. 6: IL-15 promotes differentiation of peripheral NK into ielLC1-like cells, in vivo. (A) Protocol used to evaluate the in vivo differentiation of peripheral NK cells into ILC1-like cells. In brief, 10⁶ PCI13 were subcutaneously injected in the flank of NSG mice, after 10-15 days (when the tumor were palpable) peripheral NK cells were obtained from blood and were injected intra-tumor. Mice were treated or not with IL15 (5 μg/mouse) every 2 days. After 6 days, tumors were harvested and the phenotype and function of intra-tumoral NK cells were assessed. (B) According to the protocol described in (A), sorted Lin⁻CD56⁺ NK cells were intra-tumor injected. Graph shows cumulative results of the percentage of CD49a⁺ NK cells obtained following intra-tumor injection of sorted NK cells. (C-D) According to the protocol described in (A), sorted Lin⁻CD56⁺CD94⁺CD16⁻ Stage 4b NK cells were intra-tumor injected. (C, LEFT) Dot plots show the expression of CD49a and CD103 on NK cells obtained following intra-tumor injection of sorted stage 4b NK cells, in the presence or not of IL15 in vivo. (MIDDLE) Graph shows cumulative results of the percentage of CD49a⁺ NK cells obtained following intra-tumor injection of sorted stage 4b NK cells, in the presence or not of IL15 in vivo. (RIGHT) Graph shows cumulative results of the percentage of CD49a⁺CD103⁺NK cells obtained following intra-tumor injection of sorted stage 4b NK cells, in the presence or not of IL15 in vivo. (D) Stage 4b NK cells were injected intra-tumor and were in the presence or not of IL15 in vivo, after 6 days, tumors were harvested and processed into single-cell suspensions, then single-cell suspensions were stimulated or not by PMA/lonomycin. (LEFT) Histograms show IFNg production by intra-tumoral CD49a⁻ and CD49a⁺ NK cells, in response to PMA/lonomycin or control condition (unstimulated). (MIDDLE) Graph shows cumulative results of the percentage of IFNg⁺ NK cells, in response to PMA/lonomycin or control condition (unstimulated). (RIGHT) Graph shows cumulative results of the MFI of IFNg on NK cells, in response to PMA/lonomycin or control condition (unstimulated). (E) Correlation between IL15 gene expression in human HNSCC tumors from the TGCA database and an ielLC1 gene expression signature obtained from the scRNA-seq data.

FIG. 7: Co-culture of peripheral NK cells with IL-15 and TGFbeta enhances differentiation into ielLC1-like cells. (A) Using a co-culture system similar to FIG. 5A, NK cells cells were co-cultured with MDCK (top) or PCI-13 (bottom) in the presence of IL-15 and either the TGFbeta receptor 2 inhibitor ITD1 or the ALK receptor inhibitor SB-431542 or TGFb. In these conditions, there was significant inhibition of ielLC1-like cell differentiation in the presence of TGFb signaling inhibitors and stimulation of the ielLC1-like phenotype in the presence of TGFb. These data indicate that IL-15+TGFbeta+epithelial feeder cells results in enhanced differentiation of ielLC1-like cells. (B) The co-culture of NK cells with IL-15 and TGFbeta without a feeder layer also induced ielLC1-like cells but to a lesser degree than when combined with the epithelial feeder layer as in (A).

FIG. 8: Aryl hydrocarbon receptor can modulate ielLC1-like cell differentiation. Because CD56^{bright} NK cells preferentially differentiate into ielLC1-like cells, as shown in Supplemental FIG. 14, and CD56bright NK cells express greater levels of aryl hydrocarbon receptor (AHR), we assessed AHR activity on ielLC1-like cell differentiation. The co-culture system, as in FIG. 5A (NK cells with IL-15 and PCI-13 carcinoma cells), was performed either with an AHR activating ligand (FICZ), AHR inhibitor (CH-223191) or vehicle control (DMSO). There was profound reduction of ielLC1-like differentiation with AHR inhibitor, whereas AHR activating ligand promoted the ielLC1-like differentiation.

FIG. 9: NKp44 ligand enhances ielLC1-like cell differentiation. (A) When the co-culture system, as in FIG. 5A (NK cells with IL-15), was performed with a cell line expressing a cognate ligand of NKp44, differentiation into ielLC1-like cells was observed. Note, NKp44 is an NK cell activating receptor that is also a defining marker of ielLC1s. (B) Co-culture of NK cells with PCI-13 cells and IL-15, with and without a blocking antibody to NKp44, demonstrated that inhibition of the NKp44 receptor diminished differentiation into ielLC1-like cells

FIG. 10: CAR-NK cells induced to differentiate into ielLC1-like cells have superior killing capacity. NK cells, transduced to express chimeric antigen receptor targeting CD19, were co-cultured as in FIG. 5A (NK cells with IL-15 and PCI-13 carcinoma cells). Killing against squamous cell carcinoma cells that were transduced with a truncated CD19 (so that only the extracellular portion of CD19 is expressed) was compared between conventional CAR-NK cells and ielLC1-like CAR-NK cells. The killing by the ielLC1-like CAR NK cells was superior to conventional CAR-NK cells.

FIG. 11: Feeder cell-free culture of NK cells with extracellular matrix components can induce ielLC1-like cell differentiation. Culture plates with or without E-Cadherin coating was used to culture NK cells with IL-15. The E-Cadherin coating enhanced formation of the ielLC1-like cells

FIG. 12: Cryopreserved NK cells can be recovered, expanded, and used for differentiation into ielLC1-like cells. NK cells were frozen in human AB serum+DMSO at 10 million cells/mL. Cells were later thawed, cultured with irradiated K562-21+IL-15 for 4 days, then cultured with irradiated PCI-13+IL-15 for 4 days.

FIG. 13: Gating strategy for intra-tumoral innate lymphoid cells. Gating strategy used to study intratumoral innate lymphoid cell populations. In brief, we first gated on lymphocytes according to FSC and SCC parameters, then doublets were excluded, then we gated on live CD45⁺Lin(CD3/CD5/CD14/CD19/CD20/CD34)⁻ cells, finally we gated on CD56^{+/-}CD127^{+/-} cells. The latter gate including NK cells and known subsets of ILCs.

FIG. 14: QC metrics and cut-offs for single-cell RNA-Seq analysis

FIG. 15: UMAPs with additional annotations. (A) By cell type (PBMC: peripheral innate lymphoid cells, NLN: innate lymphoid cells from normal lymph node, PRI: innate lymphoid cells from primary tumor, MET: innate lymphoid cells from metastatic tumor). (B) By surface markers on FACS sorting. (C) By source patient.

FIG. 16: Deconvoluted gene expression fractions of intratumoral innate lymphoid cells by CIBERSORTx (A) Deconvolution of intratumoral innate lymphoid cells by gene expression signatures derived from Collins et al. Cell, 2019. (B) Deconvolution of intratumoral innate lymphoid cells by gene expression signatures derived from Yudanin et al. Immunity, 2019.

FIG. 17: Expression of EOMES and TBET. (A) Violin plot showing the expression level of Eomes on the different clusters. (B) Violin plot showing the expression level of Tbet on the different clusters.

FIG. 18: Phenotypic analysis of primary intratumoral CD49a+ ielLC1 cells. (A-F) Single-cell suspensions of human HNSCC tumor specimens were stained for surface and intracellular markers. (A, LEFT) Gated on Lin-CD56^{+/-}CD127^{+/-} as described in Supplemental FIG. 1, dot plot shows the expression of CD49a and CD103. (RIGHT) Histograms show the expression of Granzyme b (Gzmb), Ki67 and CD16 on CD49a⁻CD103⁻, CD149a⁻CD103⁺, CD49a⁺CD103⁻ and CD49a⁺CD103⁺ cells. (B, LEFT) Histogram shows the expression of CD69 on CD49a+ and CD49a− cells. (RIGHT) Graph shows cumulative results of the expression of CD69 on CD49a+ and CD49a− cells. (C, LEFT) Gated on Lin⁻CD56^{+/-}CD127^{+/-} as described in Supplemental FIG. 1, dot plot shows expression of CD49a and CD94. (RIGHT) Gated on Lin⁻CD56^{+/-}CD127^{+/-} as described in Supplemental FIG. 1, dot plot shows expression of CD49a and NKp80, (D) Graph shows the percentage of CD49a+ cells on tumor samples from different patients. (E) Graph shows the percentage of CD49a+CD103+ cells on tumor samples from different patients. (F) Correlation of the percentage of CD49a+ and the percentage of CD49a+CD103+ cells, ex vivo.

FIG. 19: Expression of selected markers on primary intratumoral NK cells. (A-C) Single-cell suspensions of human HNSCC tumor specimens were stained for surface markers. (A, LEFT) Histogram shows the expression of PD1 on NK cells. (RIGHT) Graph shows cumulative results for the expression of PD1 on NK cells. (B, LEFT) Histogram shows the expression of TRAIL on NK cells, (RIGHT) Graph shows cumulative results for the expression of TRAIL on NK cells. (C, LEFT) Histogram shows the expression of NKp44 on NK cells. (RIGHT) Graph shows cumulative results for the expression of NKp44 on NK cells.

FIG. 20: Functional response of primary intratumoral NK cells, ex vivo. (A-D) Single-cell suspensions of human HNSCC tumor specimens were stimulated under different conditions during 4 hours, as described in Materials and Methods, then cells were stained for surface and intracellular markers. (A, LEFT) Histogram shows the production of IFNg by CD49a− and CD49a+ cells, in response to PMA+lono or control condition (unstimulated). (RIGHT) Graph shows cumulative results of the percentage of IFNg+ on CD49a− and CD49a+ cells, in response to PMA+lono. (B) Graph shows cumulative results of the percentage of IFNg+ on CD49a− and CD49a+ cells, in response to IL12+IL15+IL18. (C) Graph shows cumulative results of the percentage of IFNg+ on CD49a− and CD49a+ cells, in response to K562. (D) Graph shows cumulative results of the degranulation (CD107a expression) by CD49a− and CD49a+ cells, in response to K562.

FIG. 21: Functional response of primary intratumoral NK cells from anti-PD1-treated patients, ex vivo. (A-C) Single-cell suspension of HNSCC tumor specimen from patient who received anti-PD1 therapy, was stimulated under different conditions during 4 hours as described in Materials and Methods, then cells were stained for surface and intracellular markers. (A, LEFT) Histogram shows the production of IFNg by CD49a− and CD49a+ cells, in response to PMA+lono or control condition (unstimulated), (RIGHT) Graph shows percentage of IFNg+ on CD49a− and CD49a+ cells, in response to PMA+lono. (B) Histogram shows the production of IFNg by CD49a− CD103−, CD49a−CD103+; CD49a+CD103− and CD49a+CD103+ cells, in response to PMA+lono or control condition (unstimulated). (C) Graph shows percentage of IFNg+ on CD49a− and CD49a+ cells, in response to IL12+IL15+IL18.

FIG. 22: Gating strategy to sort peripheral NK cells. (A-B) Gating strategy used to sort peripheral NK cells from single-cell enriched NK. In brief, we first gated on lymphocytes according to FSC and SCC parameters, then doublets were excluded, then we gated on live Lin(CD3/CD5/CD14/CD19/CD20)^{neg}CD56^{pos} NK cells. A Shows the pre-sorted cells. B. Shows the post-sorted cells.

FIG. 23: HNSCC cells and IL15 promote differentiation of peripheral NK cells into ielLC1-like cells in vitro. (A, LEFT) Dot plot shows the expression of CD49a and CD103 in peripheral NK cells ex vivo, (RIGHT) Graph shows cumulative results of the percentages of CD49a⁻CD103⁻, CD49a⁻CD103⁺, CD49a⁺CD103⁻ and CD49a⁺CD103⁺ NK cells ex vivo. (B) Peripheral NK cells were co-cultured with SCC4 or SCC6 in the presence or not of IL15 (10 ng/mL), as described in FIG. 5A. Dot plots show the expression of CD49a and CD103 on NK cells after culture.

FIG. 24: Cytokines and chemokines secreted by PCI13 cells. PCI13 cells were cultured alone (10⁴ PCI13 per wel, in a 96-well plate) during 3 days, then supernatants were harvested and 62-plex Luminex Immuno-Assay was performed. Graph shows cytokines and chemokines secreted by PCI13, in duplicate measurements.

FIG. 25: Phenotypic analysis of in vitro differentiated ielLC1-like cells. (A-B) NK cells were cultured with PCI13 and IL15 (10 ng/mL), as described in FIG. 5A. Then, cells were harvested for phenotypic characterization. (A) Histograms show the expression of Tbet, Eomes, GzmB, Ki67, NKp44, CD94 and CD16, on CD49a⁻CD103⁻, CD49a⁻CD103⁺, CD49a⁺CD103⁻ and CD49a⁺CD103⁺ cells. (B) Dot plot shows CD49a and NKp80 expression, after in vitro differentiation of S4b NK cells.

FIG. 26: Peripheral CD56^{bright} (S4b) NK cells preferentially differentiate into ielLC1-like cells. (A, LEFT) Dot plots show the expression of CD49a and CD103, on CD56^{bright} NK cells that were cultured with PCI13 and IL15 (10 ng/mL), or control conditions, during 4 days. (MIDDLE) Graph shows cumulative results of the percentage of CD49a⁺ cells, after culture of CD56^{bright} NK cells with PCI13 and IL15 (10 ng/mL), or control conditions, during 4 days. (RIGHT) Graph shows cumulative results of the MFI of CD49a on NK cells, after culture of CD56^{bright} NK cells with PCI13 and IL15 (10 ng/mL), or control conditions, during 4 days. (B) Graph shows cumulative results of the percentage of CD49a⁺CD103⁺ cells, after culture of CD56^{bright} NK cells with PCI13 and IL15 (10 ng/mL), or control conditions, during 4 days. (C, LEFT) Dot plots show the expression of CD49a and CD103, on CD56^{dim} NK cells that were cultured with PCI13 and IL15 (10 ng/mL), or control conditions, during 4 days. (MIDDLE) Graph shows cumulative results of the percentage of CD49a⁺ cells, after culture of CD56^{dim} NK cells with PCI13 and IL15 (10 ng/mL), or control conditions, during 4 days. (RIGHT) Graph shows cumulative results of the MFI of CD49a on NK cells, after culture of CD56^{dim} NK cells with PCI13 and IL15 (10 ng/mL), or control conditions, during 4 days. (D) Graph shows cumulative results of the percentage of CD49a⁺CD103⁺ cells, after culture of CD56^{dim} NK cells with PCI13 and IL15 (10 ng/mL), or control conditions, during 4 days.

FIG. 27: IelLC1 are also found in primary melanoma. Human primary melanoma tumor tissue was analyzed for the presence of ielLC1-like cells. Dot plot shows the expression of CD49a and CD103 on intra-tumoral NK cells.

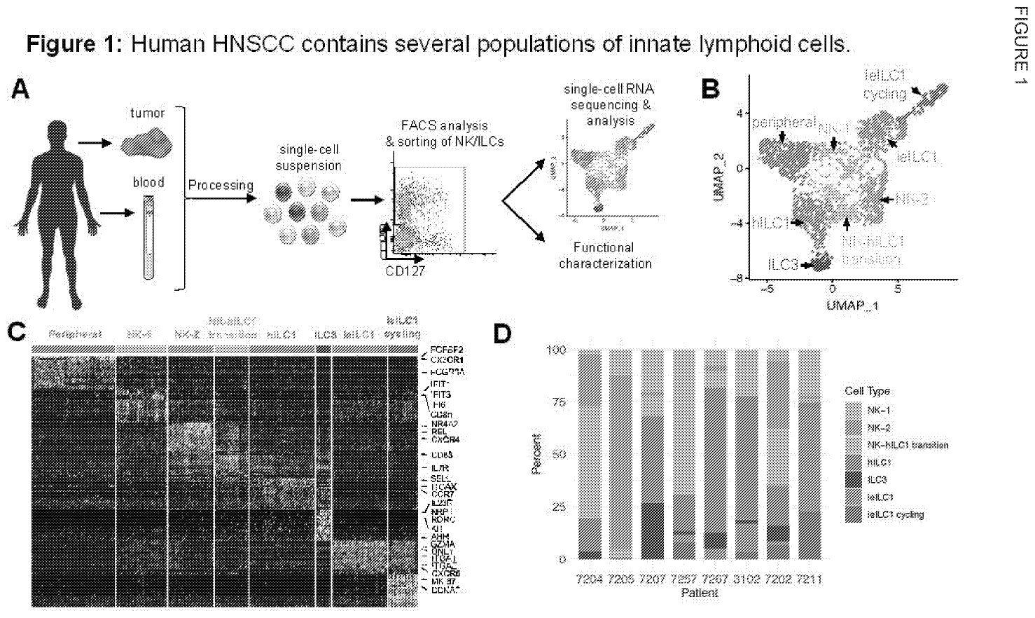

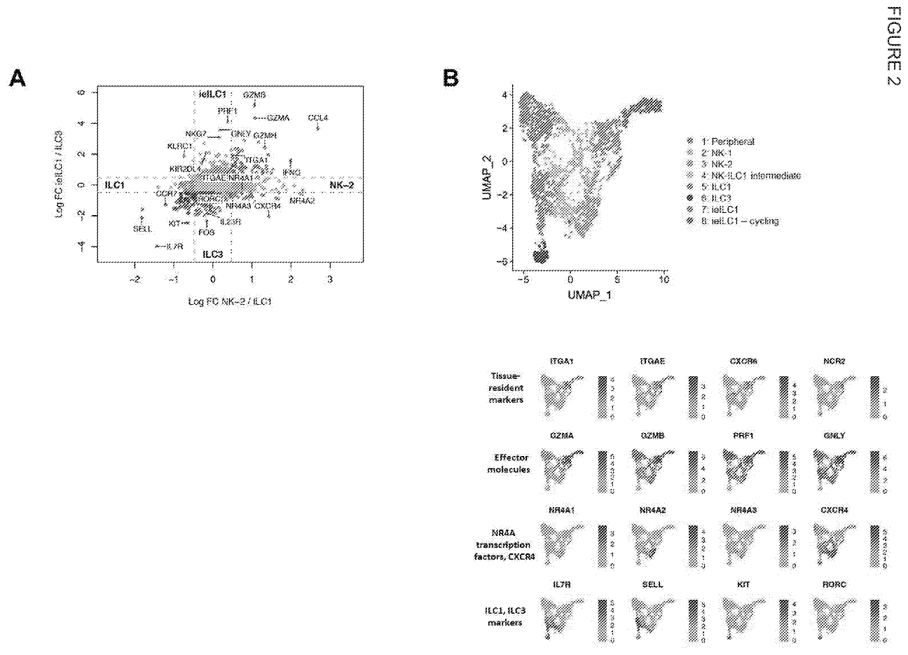

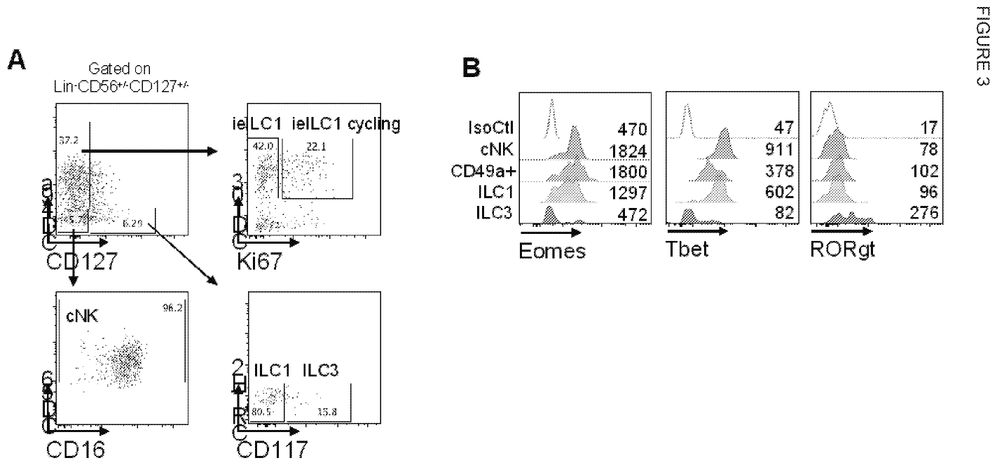

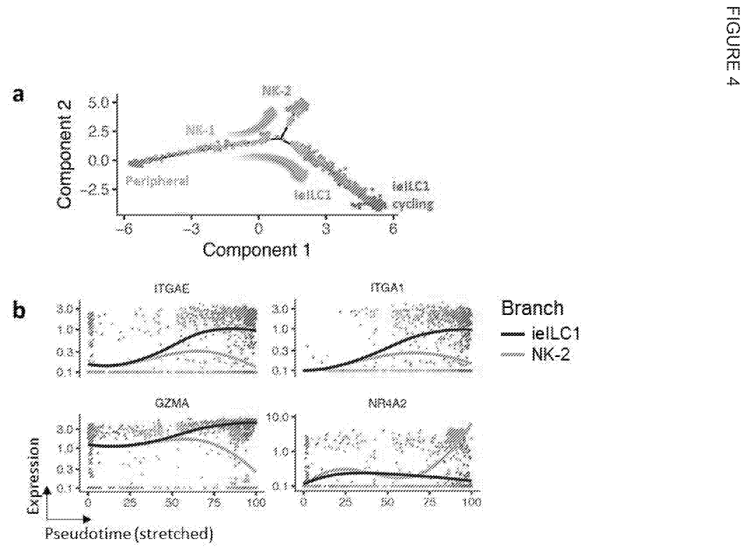

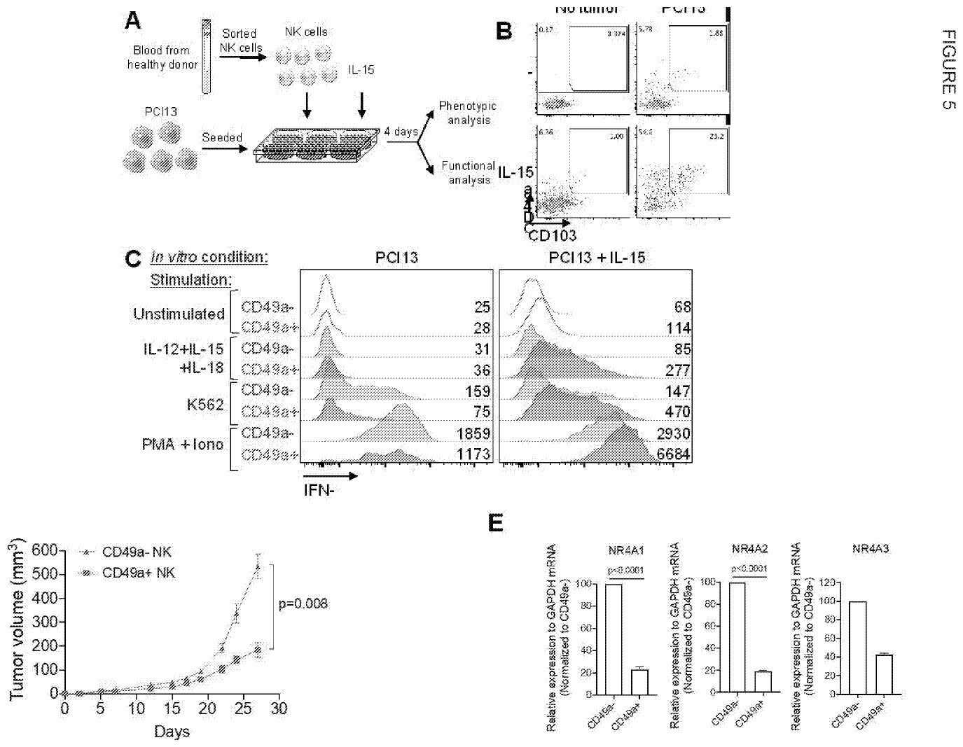

### DETAILED DESCRIPTION OF THE EMBODIMENTS

In order for the present disclosure to be more readily understood, certain terms and phrases are defined below as well as throughout the specification. The definitions provided herein are non-limiting and should be read in view of what one of skill in the art would know at the time of invention.

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It should be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Innate lymphoid cells (ILCs) are a group of innate immune cells that are derived from common lymphoid progenitor (CLP) and belong to the lymphoid lineage. These cells are defined by absence of antigen specific B or T cell receptor, and lack certain markers specific for myeloid or dendritic cell markers. This group of cells has diverse physiological functions in protective immunity and the regulation of homeostasis and inflammation

ILCs can be divided based on the cytokines that they can produce, and the transcription factors that regulate their development and function. There is a larger family of Group 1 ILCs. Within the Group 1 ILCs in humans, there are three main cell type listed here and defined as follows:
NK cell: CD56+ CD94+ Eomesodermin (EOMES)+ Tbet+ CD49a− CD103− NKp44+/− Granzyme+ Perforin+
ILC1: CD127+ CD56− CD94− EOMES− Tbet+ Granzyme− Perforin−
ielLC1: CD56+ CD94+ EOMES+ Tbet+ CD49a+ CD103+ NKp44+ CXCR6+ Granzyme+ Perforin+

The NK cells and ielLC1s are cytotoxic and produce IFNgamma. These are EOMES+.

The “classic” ILC1s are not cytotoxic but produce IFNgamma. These are EOMES−. The ielLC1s are “tissue-resident” and do not normally circulate in the blood. They express markers of tissue residency (CD49a and CD103).

Natural killer (‘NK’) cells are cytotoxic innate effector cells analogous to the cytotoxic T cells of the adaptive immune system. They are distributed throughout the blood, organs, and lymphoid tissue and make up around 15% of the peripheral blood lymphocytes. NK cells play a role in tumor surveillance and the rapid elimination of virus-infected cells.

Innate lymphoid cells (ILCs) are immune cells having features of lymphocytes, and are rapid producers of both proinflammatory and regulatory cytokines in response to local injury, inflammation, pathogen infection, or commensal microbiota perturbation. Most ILCs have been shown to be tissue resident during homeostasis.

IL-15 is 14-15 kDa glycoprotein encoded by the 34 kb region of chromosome 4q31 in humans, and at the central region of chromosome 8 in mice. The human IL-15 gene comprises nine exons (1-8 and 4A) and eight introns, four of which (exons 5 through 8) code for the mature protein. Two alternatively spliced transcript variants of this gene encoding the same protein have been reported. Although IL-15 mRNA can be found in many cells and tissues including mast cells, cancer cells or fibroblasts, this cytokine is produced as a mature protein mainly by dendritic cells, monocytes and macrophages. The main mechanism of IL-15 signaling is trans-presentation which is mediated by membrane-bound complex IL-15/IL-15Rα. IL-15 is also able to bind to the 15Rβγc signaling complex with intermediate affinity without requirement for IL-15Rα receptor. The reference sequence of the human protein is available at Genbank, accession number NP_000576; NP_751915. An effective dose of IL-15 for in vitro culture may be from about 0.1 ng/ml, from about 1 ng/ml, from about 5 ng/ml, from about 10 ng/ml, from about 25 ng/ml, or more. For in vitro cultures increased concentrations are typically not deleterious, although usually the concentration is not more than about 100-500 ng/ml. The in vivo dose of IL-15 may be from about 10 μg/kg, from about 100 μg/kg, from about 250 μg/kg, from about 500 μg/kg, from about 750 μg/kg, from about 1 mg/kg, and not more than about 10 mg/kg. The dosage may be adjusted for local or localized administration.

An effective dose of TGFβ for in vitro culture may be from about 0.1 ng/ml, from about 1 ng/ml, from about 5 ng/ml, from about 10 ng/ml, from about 25 ng/ml, or more. For in vitro cultures increased concentrations are typically not deleterious, although usually the concentration is not more than about 100-500 ng/ml. The in vivo dose of IL-15 may be from about 10 μg/kg, from about 100 μg/kg, from about 250 μg/kg, from about 500 μg/kg, from about 750 μg/kg, from about 1 mg/kg, and not more than about 10 mg/kg. The dosage may be adjusted for local or localized administration.

The terms “recipient”, “Individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. Preferably, the mammal is human.

As used herein, a “therapeutically effective amount” refers to that amount of the therapeutic agent, e.g. adoptive differentiated ielLC1-like cells, sufficient to prevent, treat or manage a disease or disorder. A therapeutically effective amount may refer to the amount of therapeutic agent sufficient to delay or minimize the onset of disease, e.g., delay or minimize the spread of cancer, or the amount effect to decrease or increase signaling from a receptor of interest. A therapeutically effective amount may also refer to the amount of the therapeutic agent that provides a therapeutic benefit in the treatment or management of a disease, e.g. by killing of cancer cells. Further, a therapeutically effective amount with respect to a therapeutic agent of the invention means the amount of therapeutic agent alone, or in combination with other therapies, that provides a therapeutic benefit in the treatment or management of a disease.

As used herein, the terms “prevent”, “preventing” and “prevention” refer to the prevention of the recurrence or onset of one or more symptoms of a disorder in a subject as result of the administration of a prophylactic or therapeutic agent.

As used herein, the term “in combination” refers to the use of more than one prophylactic and/or therapeutic agents. The use of the term “in combination” does not restrict the order in which prophylactic and/or therapeutic agents are administered to a subject with a disorder. A first prophylactic or therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent to a subject with a disorder.

NK-cells useful for differentiating by the methods described herein include peripheral blood NK cells. NK cells for differentiating as described above are collected from a subject or a donor may be separated from a mixture of cells by techniques that enrich for desired cells or may be differentiated and cultured without separation. An appropriate solution may be used for dispersion or suspension. Such solution will generally be a balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc. Techniques for affinity separation may include magnetic separation, using antibody-coated magnetic beads, affinity chromatography, and “panning” with antibody attached to a solid matrix, e.g., a plate, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g., propidium iodide). Any technique may be employed which is not unduly detrimental to the viability of the selected cells. The affinity reagents may be specific receptors or ligands for the cell surface molecules indicated above. In addition to antibody reagents, peptide-MHC antigen and T cell receptor pairs may be used; peptide ligands and receptor; effector and receptor molecules, and the like.

The separated cells may be collected in any appropriate medium that maintains the viability of the cells, usually having a cushion of serum at the bottom of the collection tube. Various media are commercially available and may be used according to the nature of the cells, including dMEM, HBSS, dPBS, RPMI, Iscove's medium, etc., frequently supplemented with fetal calf serum (FCS). The collected and optionally enriched cell population may be used immediately for genetic modification, differentiation, or expansion. The cells may also be cryopreserved at liquid nitrogen temperatures and stored, being thawed and capable of being reused. The cells will usually be stored in 10% DMSO, 50% FCS, 40% RPMI 1640 medium, but can also be stored in 10% DMSO, 90% Human AB Serum medium, or several other combinations of DMSO ranging from 1% to 20% and serum ranging from 10% to 90%, in combination with other reagents such as dextran.

In some embodiments, a differentiated cell is allogeneic with respect to the individual that is treated. In some embodiments an allogeneic differentiated ielLC1-like cell is fully HLA matched. However not all patients have a fully matched donor and a cellular product suitable for all patients independent of HLA type provides an alternative.

Following culture, the differentiated ielLC1-like cells may be isolated from the population of cultured cells, using generally methods described above. The differentiated cells may be infused to the subject in any physiologically acceptable medium by any convenient route of administration, normally intravascularly, although they may also be introduced by other routes, where the cells may find an appropriate site for growth. Usually, at least 1×10⁶ cells/kg will be administered, at least 1×10⁷ cells/kg, at least 1×10⁸ cells/kg, at least 1×10⁹ cells/kg, at least 1×10¹⁰ cells/kg, or more, usually being limited by the number of cells that are obtained.

An enhanced immune response may be manifest as an increase in the cytolytic response of the differentiated cells towards the target cancer cells present in the recipient, e.g. towards elimination of tumor cells; and the like.

Differentiated cells can be provided in pharmaceutical compositions suitable for therapeutic use, e.g. for human treatment. Therapeutic formulations comprising such cells can be frozen, or prepared for administration with physiologically acceptable carriers, excipients or stabilizers (Remington's Pharmaceutical Sciences 16th edition, Osol, A. Ed. (1980)), in the form of aqueous solutions. The cells will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The cells can be administered by any suitable means, usually parenteral. Parenteral infusions include intramuscular, intravenous (bolus or slow infusion), intraarterial, intraperitoneal, intrathecal or subcutaneous administration.

The differentiated ielLC1-like cells may be infused to the subject in any physiologically acceptable medium, normally intravascularly, although they may also be introduced into any other convenient site, where the cells may find an appropriate site for growth. Usually, at least 1×10⁶ cells/kg will be administered, at least 1×10⁷ cells/kg, at least 1×10³ cells/kg, at least 1×10⁹ cells/kg, at least 1×10¹⁰ cells/kg, or more, usually being limited by the number of T cells that are obtained during collection.

For example, typical ranges for the administration of cells for use in the practice of the present invention range from about 1×10⁵ to 5×10⁸ viable cells per kg of subject body weight per course of therapy. Consequently, adjusted for body weight, typical ranges for the administration of viable cells in human subjects ranges from approximately 1×10⁶ to approximately 1×10¹³ viable cells, alternatively from approximately 5×10⁶ to approximately 5×10¹² viable cells, alternatively from approximately 1×10⁷ to approximately 1×10¹² viable cells, alternatively from approximately 5×10⁷ to approximately 1×10¹² viable cells, alternatively from approximately 1×10⁸ to approximately 1×10¹² viable cells, alternatively from approximately 5×10⁸ to approximately 1×10¹² viable cells, alternatively from approximately 1×10⁹ to approximately 1×10¹² viable cells per course of therapy. In one embodiment, the dose of the cells is in the range of 2.5-5×10° viable cells per course of therapy.

A course of therapy may be a single dose or in multiple doses over a period of time. In some embodiments, the cells are administered in a single dose. In some embodiments, the cells are administered in two or more split doses administered over a period of 1, 2, 3, 4, 5, 6, 7, 8, 9, 10, 11, 12, 13, 14, 21, 28, 30, 60, 90, 120 or 180 days. The quantity of differentiated cells administered in such split dosing protocols may be the same in each administration or may be provided at different levels. Multi-day dosing protocols over time periods may be provided by the skilled artisan (e.g. physician) monitoring the administration of the cells taking into account the response of the subject to the treatment including adverse effects of the treatment and their modulation as discussed above.

As used herein, the terms “cancer” (or “cancerous”), “hyperproliferative,” and “neoplastic” to refer to cells having the capacity for autonomous growth (e.g., an abnormal state or condition characterized by rapidly proliferating cell growth). Hyperproliferative and neoplastic disease states may be categorized as pathologic (e.g., characterizing or constituting a disease state), or they may be categorized as non-pathologic (e.g., as a deviation from normal but not associated with a disease state). The terms are meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair. The terms “cancer” or “neoplasm” are used to refer to malignancies of the various organ systems, including those affecting the lung, breast, thyroid, lymph glands and lymphoid tissue, gastrointestinal organs, and the genitourinary tract, as well as to adenocarcinomas which are generally considered to include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. An “adenocarcinoma” refers to a carcinoma derived from glandular tissue or in which the tumor cells form recognizable glandular structures.

Examples of tumor cells include but are not limited to AML, ALL, CML, adrenal cortical cancer, anal cancer, aplastic anemia, bile duct cancer, bladder cancer, bone cancer, bone metastasis, brain cancers, central nervous system (CNS) cancers, peripheral nervous system (PNS) cancers, breast cancer, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, Hodgkin's lymphoma, Kaposi's sarcoma, kidney cancer, laryngeal and hypopharyngeal cancer, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcomas, melanoma skin cancer, non-melanoma skin cancers, stomach cancer, testicular cancer, thymus cancer, thyroid cancer, uterine cancer (e.g. uterine sarcoma), transitional cell carcinoma, vaginal cancer, vulvar cancer, mesothelioma, squamous cell or epidermoid carcinoma, bronchial adenoma, choriocarinoma, head and neck cancers, teratocarcinoma, or Waldenstrom's macroglobulinemia.

The compositions and method of the present invention may be combined with additional therapeutic agents, For example, when the disease, disorder or condition to be treated is a neoplastic disease (e.g, cancer) the methods of the present invention may be combined with conventional chemotherapeutic agents or other biological anti-cancer drugs such as checkpoint inhibitors (e.g. PD1 or PDL1 inhibitors) or therapeutic monoclonal antibodies (e.g Avastin, Herceptin).

Examples of chemical agents identified in the art as useful in the treatment of neoplastic disease, include without limitation, abitrexate, adriamycin, adrucil, amsacrine, asparaginase, anthracyclines, azacitidine, azathioprine, bicnu, blenoxane, busulfan, bleomycin, camptosar, camptothecins, carboplatin, carmustine, cerubidine, chlorambucil, cisplatin, cladribine, cosmegen, cytarabine, cytosar, cyclophosphamide, cytoxan, dactinomycin, docetaxel, doxorubicin, daunorubicin, ellence, elspar, epirubicin, etoposide, fludarabine, fluorouracil, fludara, gemcitabine, gemzar, hycamtin, hydroxyurea, hydrea, idamycin, idarubicin, ifosfamide, ifex, irinotecan, lanvis, leukeran, leustatin, matulane, mechlorethamine, mercaptopurine, methotrexate, mitomycin, mitoxantrone, mithramycin, mutamycin, myleran, mylosar, navelbine, nipent, novantrone, oncovin, oxaliplatin, paclitaxel, paraplatin, pentostatin, platinol, plicamycin, procarbazine, purinethol, ralitrexed, taxotere, taxol, teniposide, thioguanine, tomudex, topotecan, valrubicin, velban, vepesid, vinblastine, vindesine, vincristine, vinorelbine, VP-16, and vumon.

Targeted therapeutics that can be administered in combination may include, without limitation, tyrosine-kinase inhibitors, such as lmatinib mesylate (Gleevec, also known as STI-571), Gefitinib (Iressa, also known as ZD1839), Erlotinib (marketed as Tarceva), Sorafenib (Nexavar), Sunitinib (Sutent), Dasatinib (Sprycel), Lapatinib (Tykerb), Nilotinib (Tasigna), and Bortezomib (Velcade), Jakafi (ruxolitinib); Janus kinase inhibitors, such as tofacitinib; ALK inhibitors, such as crizotinib; Bcl-2 inhibitors, such as obatoclax, venclexta, and gossypol; FLT3 inhibitors, such as midostaurin (Rydapt), IDH inhibitors, such as AG-221, PARP inhibitors, such as Iniparib and Olaparib; PI3K inhibitors, such as perifosine; VEGF Receptor 2 inhibitors, such as Apatinib; AN-152 (AEZS-108) doxorubicin linked to [D-Lys(6)]-LHRH; Braf inhibitors, such as vemurafenib, dabrafenib, and LGX818; MEK inhibitors, such as trametinib; CDK inhibitors, such as PD-0332991 and LEE011; Hsp90 inhibitors, such as salinomycin; and/or small molecule drug conjugates, such as Vintafolide; serine/threonine kinase inhibitors, such as Temsirolimus (Torisel), Everolimus (Afinitor), Vemurafenib (Zelboraf), Trametinib (Mekinist), and Dabrafenib (Tafinlar).

Examples of biological agents identified in the art as useful in the treatment of neoplastic disease, include without limitation, cytokines or cytokine antagonists such as IL-12, IFNα, or anti-epidermal growth factor receptor, radiotherapy, irinotecan; tetrahydrofolate antimetabolites such as pemetrexed; antibodies against tumor antigens, a complex of a monoclonal antibody and toxin, a T-cell adjuvant, bone marrow transplant, or antigen presenting cells (e.g., dendritic cell therapy), anti-tumor vaccines, replication competent viruses, signal transduction inhibitors (e.g., Gleevec® or Herceptin®) or an immunomodulator to achieve additive or synergistic suppression of tumor growth, cyclooxygenase-2 (COX-2) inhibitors, steroids, TNF antagonists (e.g., Remicade® and Enbrel®), interferon-β1a (Avonex®), and interferon-β1b (Betaseron®) as well as combinations of one or more of the foregoing as practiced in known chemotherapeutic treatment regimens readily appreciated by the skilled clinician in the art.

Tumor specific monoclonal antibodies that can be administered in combination with differentiated ielLC1-like cells may include, without limitation, Rituximab (marketed as MabThera or Rituxan), Alemtuzumab, Panitumumab, 1pilimumab (Yervoy), etc.

In some embodiments the compositions and methods of the present invention may be combined with immune checkpoint therapy. Examples of immune checkpoint therapies include inhibitors of the binding of PD1 to PDL1 and/or PDL2. PD1 to PDL1 and/or PDL2 inhibitors are well known in the art. Examples of commercially available monoclonal antibodies that interfere with the binding of PD1 to PDL1 and/or PDL2 include nivolumab (Opdivo®, BMS-936558, MDX1106, commercially available from BristolMyers Squibb, Princeton N.J.), pembrolizumab (Keytruda®MK-3475, lambrolizumab, commercially available from Merck and Company, Kenilworth N.J.), and atezolizumab (Tecentriq®, Genentech/Roche, South San Francisco Calif.). Additional examples of PD1 inhibitory antibodies include but are not limited to durvalumab (MEDI4736, Medimmune/AstraZeneca), pidilizumab (CT-011, CureTech), PDR001 (Novartis), BMS-936559 (MDX1105, Bristol Myers Squibb), and avelumab (MSB0010718C, Merck Serono/Pfizer) and SHR-1210 (Incyte). Additional antibody PD1 pathway inhibitors are described in U.S. Pat. No. 8,217,149 (Genentech, Inc) issued Jul. 10, 2012; U.S. Pat. No. 8,168,757 (Merck Sharp and Dohme Corp.) issued May 1, 2012, U.S. Pat. No. 8,008,449 (Medarex) issued August 30, 2011, U.S. Pat. No. 7,943,743 (Medarex, Inc) issued May 17, 2011. Additionally, small molecule PD1 to PDL1 and/or PDL2 inhibitors are known in the art. See, e.g. Sasikumar, et al as WO2016142833A1 and Sasikumar, et al. WO2016142886A2, BMS-1166 and BMS-1001 (Skalniak, et al (2017) Oncotarget 8(42): 72167-72181).

In some embodiments, NK cells are modified by introduction of a CAR construct, either before or after differentiation. CARs useful in the practice of the present invention can be specific for a tumor cell antigen of interest. CARS typically comprise an antigen receptor, a transmembrane spanning domain, and a cytoplasmic domain. The antigen receptor may be specific for a tumor cell antigen, including without limitation CD19, CD123, Lewis Y, BCMA, CS1, ERBB2, IL13Ra2, EGFRvIII, gD2, mesothelin, Muc16, Muc1, and ROR1, ErbB1, CD20, PSCA, CD22, etc.

The transmembrane spanning domain of a CAR may be derived from the transmembrane domain of a naturally occurring membrane spanning protein or may be synthetic. In designing synthetic transmembrane domains, amino acids favoring alpha-helical structures are preferred. Transmembrane domains useful in construction of CARs are comprised of approximately 10, 11, 12, 13, 14, 15, 16, 17, 18, 19, 20, 21, 22, 22, 23, or 24 amino acids favoring the formation having an alpha-helical secondary structure. Amino acids having a to favor alpha-helical conformations are well known in the art. See, e.g Pace, et al, (1998) Biophysical Journal 75: 422-427. Amino acids that are particularly favored in alpha helical conformations include methionine, alanine, leucine, glutamate, and lysine. In some embodiments, the CAR transmembrane domain may be derived from the transmembrane domain from type I membrane spanning proteins, such as CD3ζ, CD4, CD8, CD28, etc.

The cytoplasmic domain of the CAR polypeptide comprises one or more intracellular signal domains. In one embodiment, the intracellular signal domains comprise the cytoplasmic sequences of the T-cell receptor (TCR) and co-receptors that initiate signal transduction following antigen receptor engagement and functional derivatives and sub-fragments thereof. A cytoplasmic signaling domain, such as those derived from the T cell receptor ζ-chain, is employed as part of the CAR in order to produce stimulatory signals for T lymphocyte proliferation and effector function following engagement of the chimeric receptor with the target antigen. Examples of cytoplasmic signaling domains include but are not limited to the cytoplasmic domain of CD27, the cytoplasmic domain S of CD28, the cytoplasmic domain of CD137 (also referred to as 4-1BB and TNFRSF9), the cytoplasmic domain of CD278 (also referred to as ICOS), p110α, β, or δ catalytic subunit of PI3 kinase, the human CD3 ζ-chain, cytoplasmic domain of CD134 (also referred to as OX40 and TNFRSF4), FcεR1γ and β chains, MB1 (Igα) chain, B29 (Igβ) chain, etc.), CD3 polypeptides (δ, Δ and ε), syk family tyrosine kinases (Syk, ZAP 70, etc.), src family tyrosine kinases (Lck, Fyn, Lyn, etc.) and other molecules involved in T-cell transduction, such as CD2, CD5 and CD28.

In some embodiments, the CAR may also provide a co-stimulatory domain. The term “co-stimulatory domain”, refers to a stimulatory domain, typically an endodomain, of a CAR that provides a secondary non-specific activation mechanism through which a primary specific stimulation is propagated. The co-stimulatory domain refers to the portion of the CAR which enhances the proliferation, survival or development of memory cells. Examples of co-stimulation include antigen nonspecific T cell co-stimulation following antigen specific signaling through the T cell receptor and antigen nonspecific B cell co-stimulation following signaling through the B cell receptor. Co-stimulation, e.g., T cell co-stimulation, and the factors involved have been described in Chen & Flies. (2013) Nat Rev Immunol 13(4):227-42. In some embodiments of the present disclosure, the CSD comprises one or more of members of the TNFR superfamily, CD28, CD137 (4-1BB), CD134 (OX40), Dap10, CD27, CD2, CD5, ICAM-1, LFA-1 (CD11a/CD18), Lck, TNFR-I, TNFR-II, Fas, CD30, CD40 or combinations thereof.

CARs are often referred to as first, second, third or fourth generation. The term first-generation CAR refers to a CAR wherein the cytoplasmic domain transmits the signal from antigen binding through only a single signaling domain, for example a signaling domain derived from the high-affinity receptor for IgE FcεRlγ, or the CD3ζchain. The domain contains one or three immunoreceptor tyrosine-based activating motif(s) [ITAM(s)] for antigen-dependent T-cell activation. The ITAM-based activating signal endows NK-cells with the ability to lyse the target tumor cells and secret cytokines in response to antigen binding. Second-generation CARs include a co-stimulatory signal in addition to the CD3ζ signal. Coincidental delivery of the delivered co-stimulatory signal enhances cytokine secretion and antitumor activity induced by CAR-transduced NK-cells. The co-stimulatory domain is usually be membrane proximal relative to the CD3ζ domain, Third-generation CARs include a tripartite signaling domain, comprising for example a CD28, CD3ζ, OX40 or 4-1BB signaling region. In fourth generation, or “armored car” CAR NK-cells are further gene modified to express or block molecules and/or receptors to enhance immune activity.

Examples of intracellular signaling domains comprising may be incorporated into the CAR include (amino to carboxy): CD3ζ; CD28-41BB-CD3ζ; CD28-OX40-CD3ζ; CD28-41BB-CD3ζ; 41BB-CD-28-CD3ζ and 41BB-CD3.

The term CAR includes CAR variants including but not limited split CARs, ON-switch CARS, bispecific or tandem CARs, inhibitory CARs (iCARs) and induced pluripotent stem (iPS) CAR-NK cells.

The term “Split CARs” refers to CARs wherein the extracellular portion, the ABD and the cytoplasmic signaling domain of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled. CAR molecules and derivatives thereof (i.e., CAR variants) are described, e.g., in PCT Application Nos. US2014/016527, US1996/017060, US2013/063083; Fedorov et al. Sci Transl Med (2013); 5(215):215ra172; Glienke et al. Front Pharmacol (2015) 6:21; Kakarla & Gottschalk 52 Cancer J (2014) 20(2):151-5; Riddell et al. Cancer J (2014) 20(2):141-4; Pegram et al. Cancer J (2014) 20(2):127-33; Cheadle et al. Immunol Rev (2014) 257(1):91-106; Barrett et al. Annu Rev Med (2014) 65:333-47; Sadelain et al. Cancer Discov (2013) 3(4):388-98; Cartellieri et al., J Biomed Biotechnol (2010) 956304; the disclosures of which are incorporated herein by reference in their entirety.

The term “bispecific or tandem CARs” refers to CARs which include a secondary CAR binding domain that can either amplify or inhibit the activity of a primary CAR.

The term “inhibitory chimeric antigen receptors” or “iCARs” are used interchangeably herein to refer to a CAR where binding iCARs use the dual antigen targeting to shut down the activation of an active CAR through the engagement of a second suppressive receptor equipped with inhibitory signaling domains of a secondary CAR binding domain results in inhibition of primary CAR activation. Inhibitory CARs (iCARs) are designed to regulate CAR-NK cells activity through inhibitory receptors signaling modules activation. This approach combines the activity of two CARs, one of which generates dominant negative signals limiting the responses of CAR-NK cells activated by the activating receptor. iCARs can switch off the response of the counteracting activator CAR when bound to a specific antigen expressed only by normal tissues. In this way, iCARs-T cells can distinguish cancer cells from healthy ones, and reversibly block functionalities of transduced T cells in an antigen-selective fashion. CTLA-4 or PD-1 intracellular domains in iCARs trigger inhibitory signals on T lymphocytes, leading to less cytokine production, less efficient target cell lysis, and altered lymphocyte motility.

The term “tandem CAR” or “TanCAR” refers to CARs which mediate bispecific activation of T cells through the engagement of two chimeric receptors designed to deliver stimulatory or costimulatory signals in response to an independent engagement of two different tumor associated antigens.

Typically, the chimeric antigen receptor NK-cells (CAR-NK cells) are NK-cells which have been recombinantly modified by transduction with an expression vector encoding a CAR in substantial accordance with the teaching above.

Cells may be prepared using the patient's own NK-cells for engineering. Consequently, the population of the cells to be administered is to the subject is necessarily variable. Additionally, since the CAR-NK cell agent is variable, the response to such agents can vary and thus involves the ongoing monitoring and management of therapy related toxicities which are managed with a course of pharmacologic immunosuppression or B cell depletion prior to the administration of the CAR-NK cell treatment. Examples of such immunosuppressive regimens including systemic corticosteroids (e.g., methylprednisolone). Therapies for B cell depletion include intravenous immunoglobulin (IVIG) by established clinical dosing guidelines to restore normal levels of serum immunoglobulin levels. In some embodiments, prior to administration of the CAR-NK cell therapy of the present invention, the subject may optionally be subjected to a lymphodepleting regimen. One example of a such lymphodepleting regimen consists of the administration to the subject of fludarabine (30 mg/m²intravenous [IV] daily for 4 days) and cyclophosphamide (500 mg/m² IV daily for 2 days starting with the first dose of fludarabine).

To achieve expression of the recombinant protein, a nucleic acid encoding an orthogonal protein (and/or CAR) is inserted into a replicable vector for expression. Many such vectors are available. The vector components generally include, but are not limited to, one or more of the following: an origin of replication, one or more marker genes, an enhancer element, a promoter, and a transcription termination sequence. Vectors include viral vectors, plasmid vectors, integrating vectors, and the like.

Expression vectors for expression of the orthogonal receptor and optionally CAR in the NK-cell may be viral vectors or non-viral vectors. Plasmids are examples of non-viral vectors. In order to facilitate transfection of the target cells, the target cell may be exposed directly with the non-viral vector may under conditions that facilitate uptake of the non-viral vector. Examples of conditions which facilitate uptake of foreign nucleic acid by mammalian cells are well known in the art and include but are not limited to chemical means (such as Lipofectamine®, Thermo-Fisher Scientific), high salt, and magnetic fields (electroporation).

In one embodiment, a non-viral vector may be provided in a non-viral delivery system.

Non-viral delivery systems are typically complexes to facilitate transduction of the target cell with a nucleic acid cargo wherein the nucleic acid is complexed with agents such as cationic lipids (DOTAP, DOTMA), surfactants, biologicals (gelatin, chitosan), metals (gold, magnetic iron) and synthetic polymers (PLG, PEI, PAMAM). Numerous embodiments of non-viral delivery systems are well known in the art including lipidic vector systems (Lee et al. (1997) Crit Rev Ther Drug Carrier Syst. 14:173-206); polymer coated liposomes (Marin et al., U.S. Pat. No. 5,213,804, issued May 25, 1993; Woodle, et al., U.S. Pat. No. 5,013,556, issued May 7, 1991); cationic liposomes (Epand et al., U.S. Pat. No. 5,283,185, issued Feb. 1, 1994; Jessee, J. A., U.S. Pat. No. 5,578,475, issued Nov. 26, 1996; Rose et al, U.S. Pat. No. 5,279,833, issued Jan. 18, 1994; Gebeyehu et al., U.S. Pat. No. 5,334,761, issued Aug. 2, 1994).

In another embodiment, the expression vector may be a viral vector. When a viral vector system is to be employed for CAR and expression of the orthogonal receptor retroviral or lentiviral expression vectors are preferred. In particular, the viral vector is a gamma retrovirus (. (Pule, et al. (2008) Nature Medicine 14(11)1264-1270), self-inactivating lentiviral vectors (June et al. (2009) Nat Rev Immunol 9(10):704-716) and retroviral vectors as described in Naldini, et al. (1996) Science 272: 263-267; Naldini, et al. (1996) Proc. Natl. Acad. Sci. USA Vol. 93, pp. 11382-11388; Dull, et al. (1998) J. Virology 72(11):8463-8471; Milone, et al, (2009) 17(8):1453-1464; Kingsman, et al. U.S. Pat. No 6,096,538 issued Aug. 1, 2000 and Kingsman, et at. U.S. Pat. No. 6,924,123 issued Aug. 2, 2005. In one embodiment of the invention, the CAR expression vector is a Lentivector® lentiviral vector available from Oxford Biomedica.

Transduction of NK-cells with an expression vector may be accomplished using techniques well known in the art including but not limited co-incubation with host NK-cells with viral vectors, electroporation, and/or chemically enhanced delivery.

Where the contacting is performed in vivo, an effective dose of engineered cells, including without limitation CAR-NK cells modified to express an orthogonal IL-2β receptor, are infused to the recipient, in combination with or prior to administration of the orthogonal cytokine, e.g. IL-2 and allowed to contact NK cells in their native environment, e.g. in lymph nodes, etc. Dosage and frequency may vary depending on the agent; mode of administration; nature of the cytokine; and the like. It will be understood by one of skill in the art that such guidelines will be adjusted for the individual circumstances. The dosage may also be varied for localized administration, e.g. intranasal, inhalation, etc., or for systemic administration, e.g. i.m., i.p., i.v., and the like. Generally at least about 10⁴ engineered cells/kg are administered, at least about 10⁵ engineered cells/kg; at least about 10⁶ engineered cells/kg, at least about 10⁷ engineered cells/kg, or more.

An enhanced immune response may be manifest as an increase in the cytolytic response of NK cells towards the target cells present in the recipient, e.g. towards elimination of tumor cells, infected cells; decrease in symptoms of autoimmune disease; and the like.

Engineered NK cells can be provided in pharmaceutical compositions suitable for therapeutic use, e.g. for human treatment. Therapeutic formulations comprising such cells can be frozen, or prepared for administration with physiologically acceptable carriers, excipients or stabilizers (Remington's Pharmaceutical Sciences 16th edition, Osol, A. Ed. (1980)), in the form of aqueous solutions. The cells will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The cells can be administered by any suitable means, usually parenteral. Parenteral infusions include intramuscular, intravenous (bolus or slow infusion), intraarterial, intraperitoneal, intrathecal or subcutaneous administration.

The engineered NK cells may be infused to the subject in any physiologically acceptable medium, normally intravascularly, although they may also be introduced into any other convenient site, where the cells may find an appropriate site for growth. Usually, at least 1×10⁶ cells/kg will be administered, at least 1×10⁷ cells/kg, at least 1×10⁸ cells/kg, at least 1×10⁹ cells/kg, at least 1×10¹⁰ cells/kg, or more, usually being limited by the number of T cells that are obtained during collection.

For example, typical ranges for the administration of cells for use in the practice of the present invention range from about 1×10⁵ to 5×10⁸ viable cells per kg of subject body weight per course of therapy. Consequently, adjusted for body weight, typical ranges for the administration of viable cells in human subjects ranges from approximately 1×10⁶ to approximately 1×10¹³ viable cells, alternatively from approximately 5×10⁶ to approximately 5×10¹² viable cells, alternatively from approximately 1×10⁷ to approximately 1×10¹² viable cells, alternatively from approximately 5×10⁷ to approximately 1×10¹² viable cells, alternatively from approximately 1×10⁸ to approximately 1×10¹² viable cells, alternatively from approximately 5×10⁸ to approximately 1×10¹² viable cells, alternatively from approximately 1×10⁹ to approximately 1×10¹² viable cells per course of therapy. In one embodiment, the dose of the cells is in the range of 2.5-5×10⁹ viable cells per course of therapy.

A course of therapy may be a single dose or in multiple doses over a period of time. In some embodiments, the cells are administered in a single dose. In some embodiments, the cells are administered in two or more split doses administered over a period of 1, 2, 3, 4, 5, 6, 7, 8, 9, 10, 11, 12, 13, 14, 21, 28, 30, 60, 90, 120 or 180 days. The quantity of engineered cells administered in such split dosing protocols may be the same in each administration or may be provided at different levels. Multi-day dosing protocols over time periods may be provided by the skilled artisan (e.g. physician) monitoring the administration of the cells taking into account the response of the subject to the treatment including adverse effects of the treatment and their modulation as discussed above.

Also provided are kits for use in the methods. The subject kits may include suitable culture kits, e.g. IL-15, a cancer cell line, etc. Kits may comprise affinity agents for selection of NK cells, and/or the selection of ielLC1-like cells. In some embodiments, the components are provided in a dosage form (e.g., a therapeutically effective dosage form), in liquid or solid form in any convenient packaging (e.g., stick pack, dose pack, etc.).

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a removed site.

The invention now being fully described, it will be apparent to one of ordinary skill in the art that various changes and modifications can be made without departing from the spirit or scope of the invention.

### EXPERIMENTAL

### Differentiating Innate Lymphoid Cells for Immunotherapy

We developed a method to in vitro differentiate peripheral conventional NK cells [Lineage(CD3/CD5/CD14/CD19/CD20)^{neg}CD56^{pos} lymphocytes] into intra-epithelial innate lymphoid cells 1 (ielLC1)-like cells, which display significant anti-tumor effector functions. As shown herein, the enhanced tumor killing activity is translated into improved in vivo activity.

In a summary of the protocol for in vitro differentiation of peripheral conventional NK cells into ielLC1-like cells: sorted, enriched, or expanded peripheral NK cells in the presence of IL15 are seeded into culture with cells such as HNSCC, which may be irradiated, or onto plate surfaces that mimic select attributes of these cells. After a suitable period of time for culture, e.g. about 4 days, the differentiated cells are harvested for analysis and effector functions.

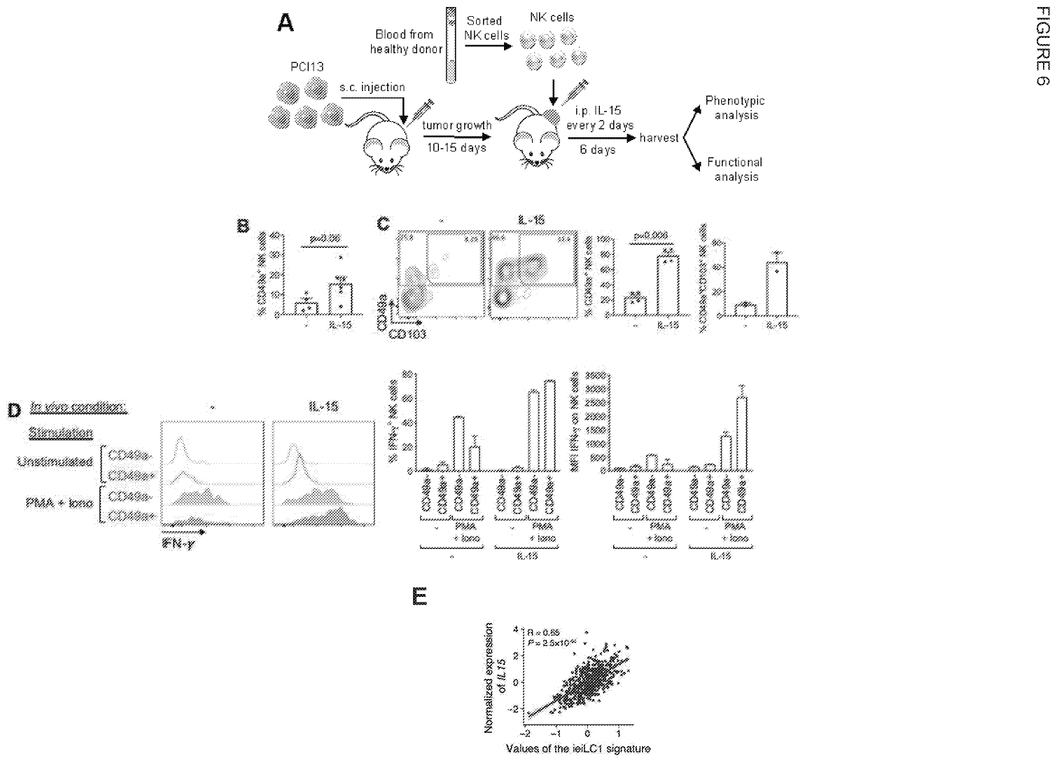

ielLC1 cells are characterized by the expression of CD49a and CD103. We show that in the presence of HNSCC cells (here PCI13) and IL-15, there is a population of cells in the co-culture that are CD49a⁺CD103⁺. As shown in FIG. 5B, after culture of NK cells with PCI13 and IL15 (10 ng/mL) or control conditions, the expression of CD49a and CD103 was analyzed on gated Lin⁻CD45⁺CD56⁻ NK cells, (LEFT) Dot plots show the expression of CD49a and CD103. (MIDDLE) Graph shows cumulative results of the percentage of CD49a⁺ on NK cells. (RIGHT) Graph shows cumulative results of the percentage of CD49a⁺CD103⁺ on NK cells.

This method, using epithelial carcinoma cells (e.g. head and neck squamous cell carcinoma), is significantly better at inducing ielLC1-like cells than existing methods to activate NK cells using irradiated K562 cells (10.7% desired phenotype v 3.29%). As shown in FIG. 5C, we can see that after in vitro differentiation, only in the presence of PCI13+IL-15, ielLC1-like cells (here represented as CD49a⁺ cells) display higher response to several types of in vitro stimulation, such as cytokine stimulation (IL-12+IL-15+IL-18), tumor cells (K562) and PMA+lonomycin.

Shown in FIG. 5D, it can be seen that after in vitro differentiation in the presence of PCI13+IL-15, ielLC1-like cells (here CD49a⁺ cells) better control tumor growth in vivo.

In addition to the protocol described here, which allows the differentiation of conventional NK cells into highly functional ielLC1-like cells with anti-tumor potential, we note that this protocol can be used in combination with existing activation/expansion protocols in order to yield high scale production of NK cells for therapeutic purposes.

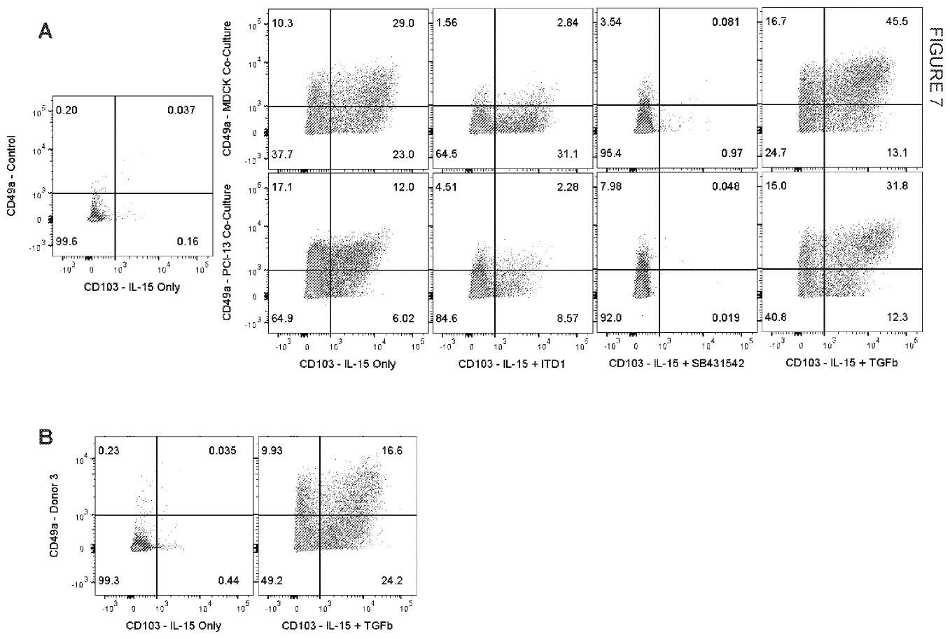

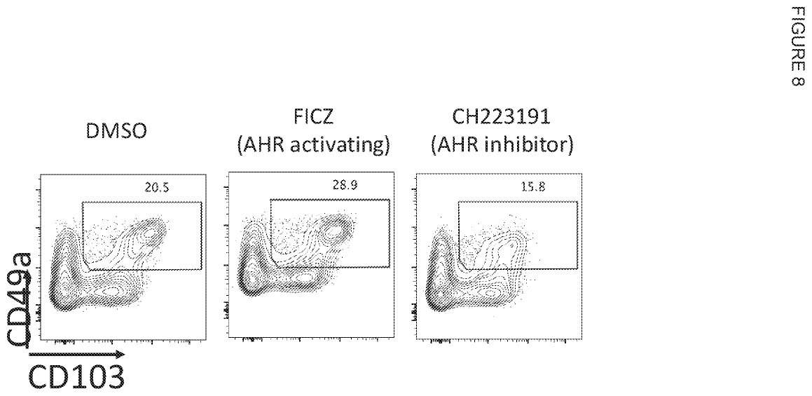

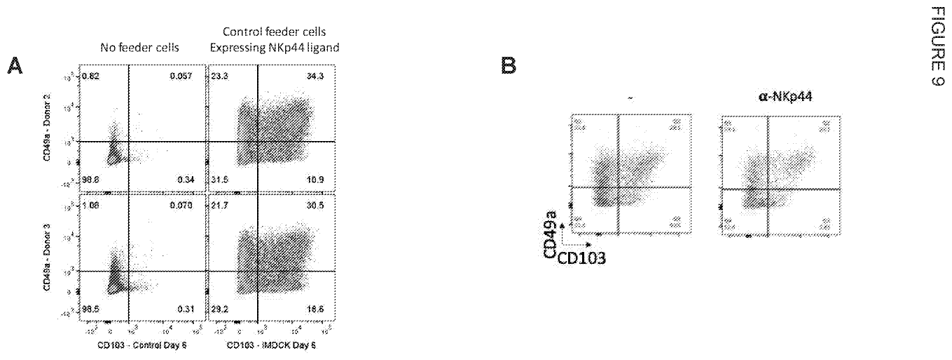

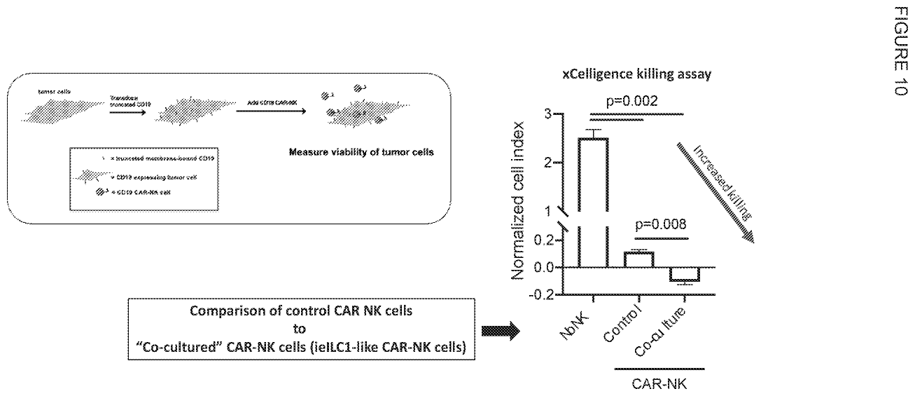

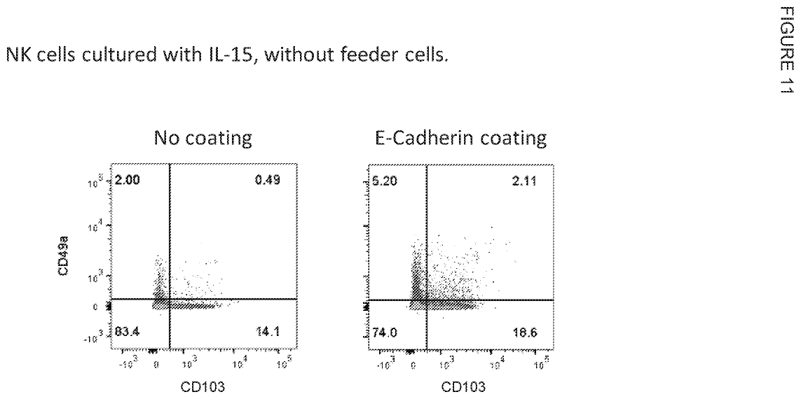

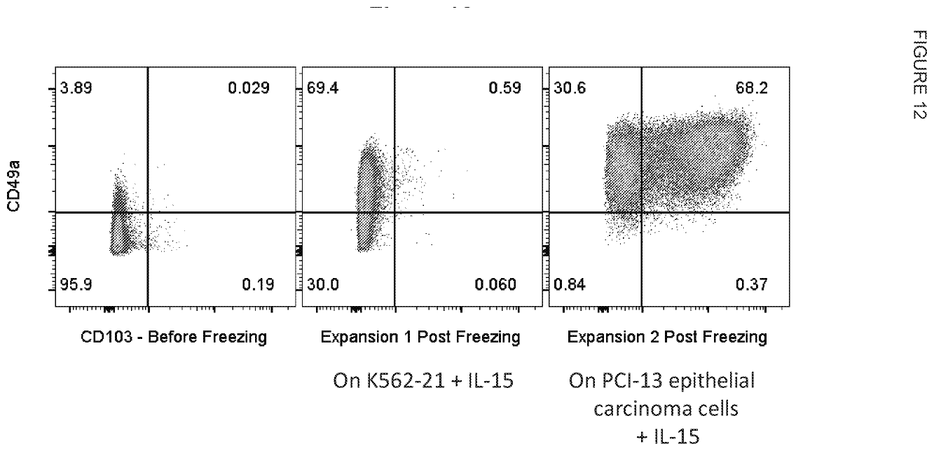

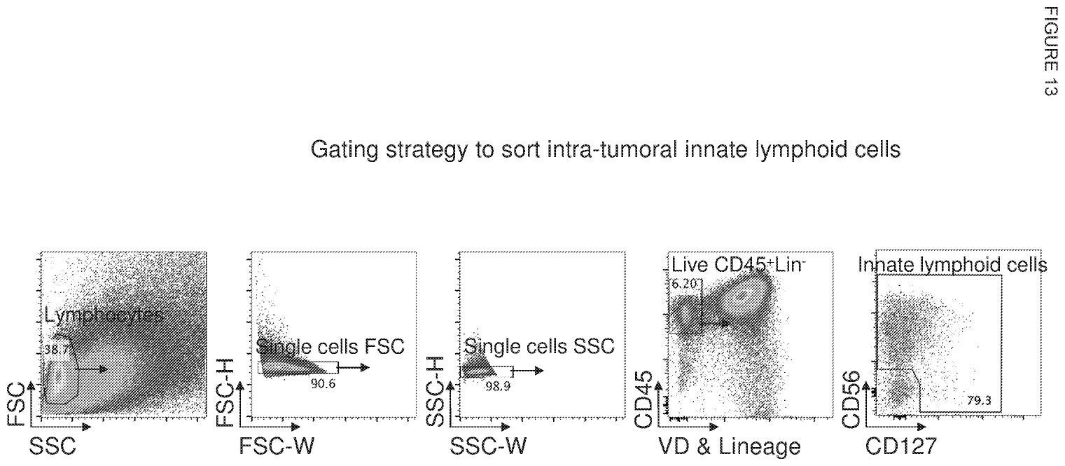

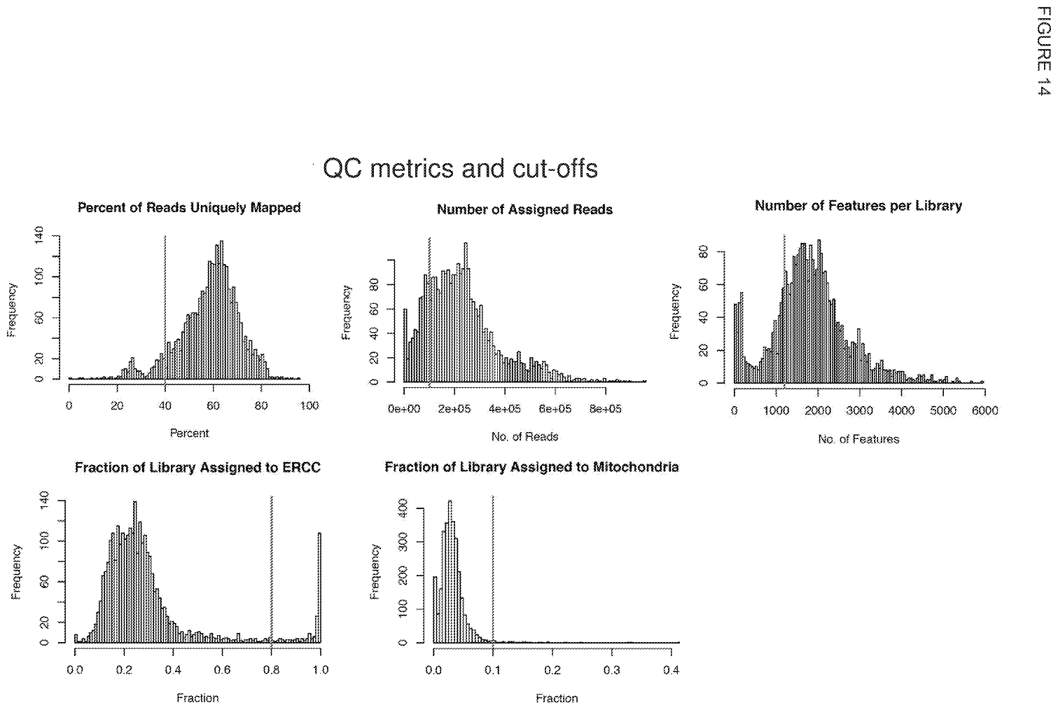

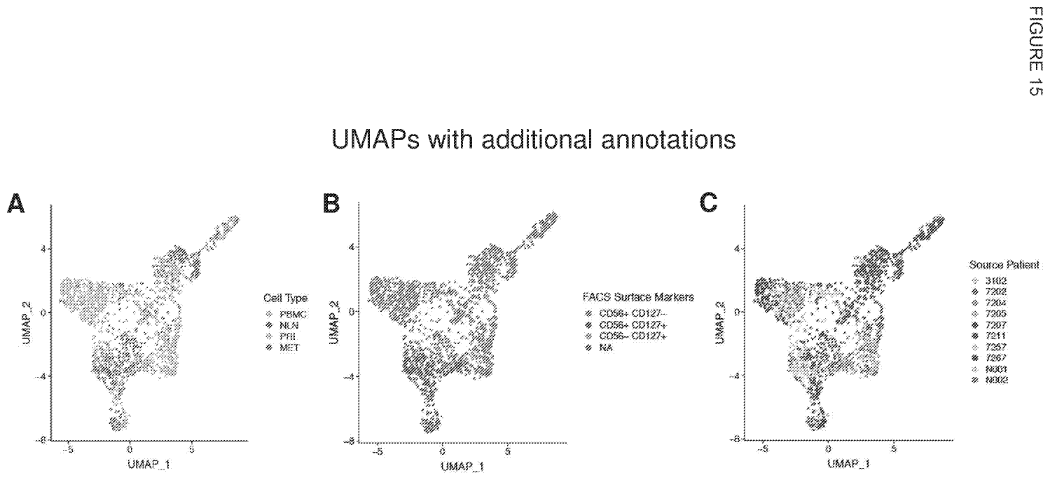

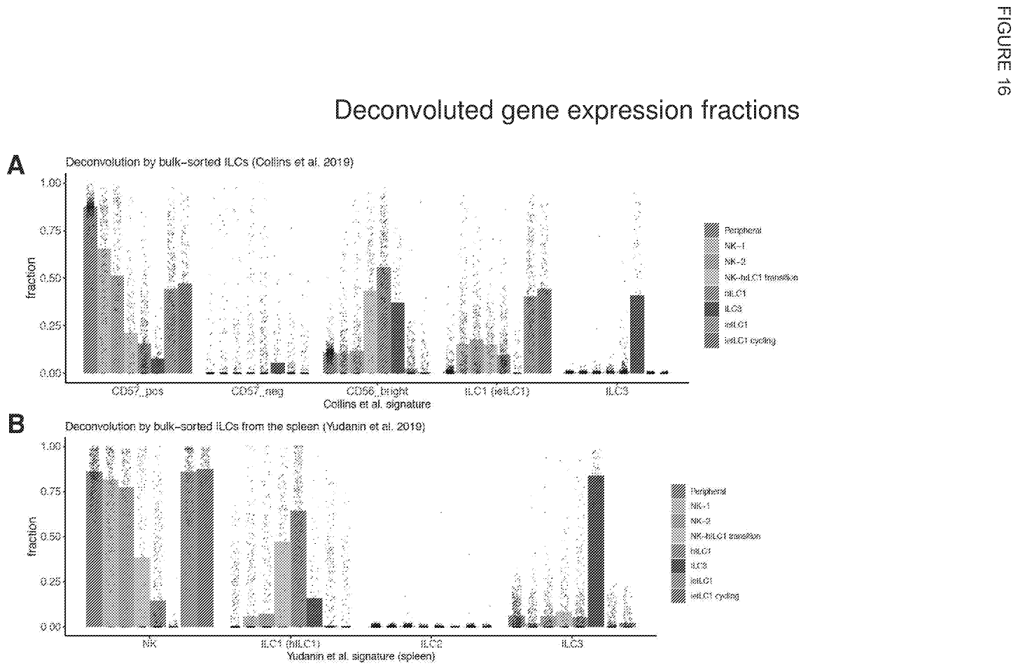

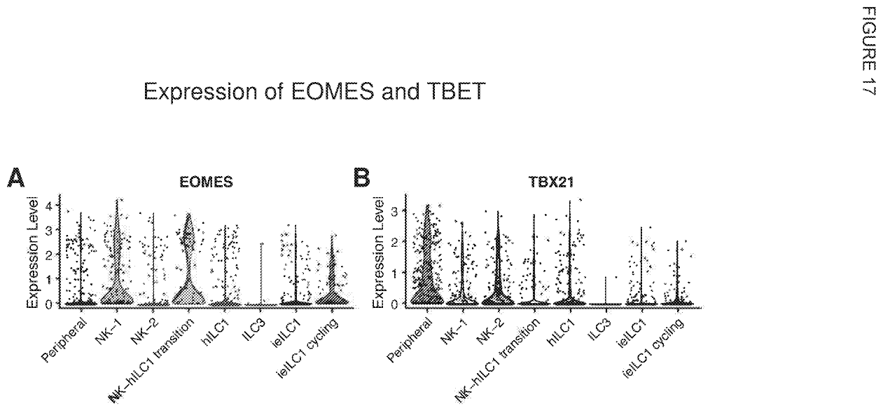

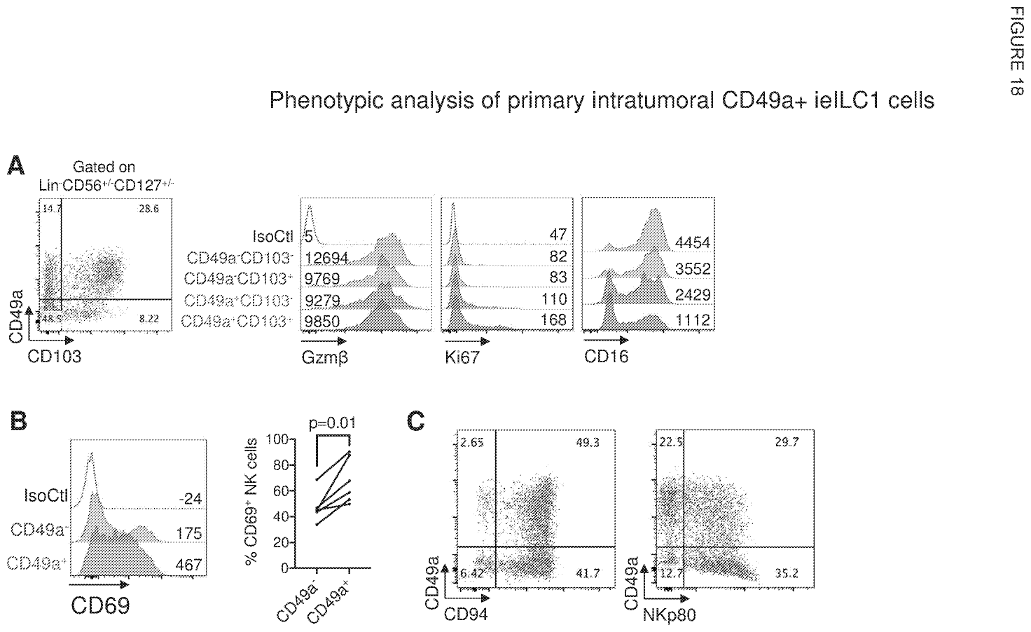

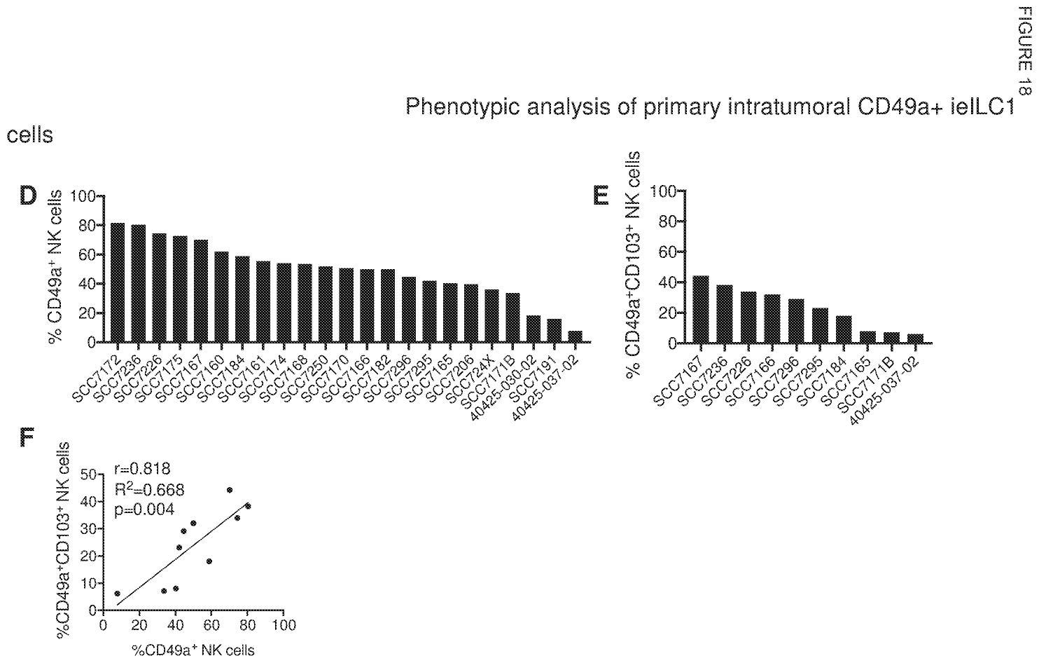

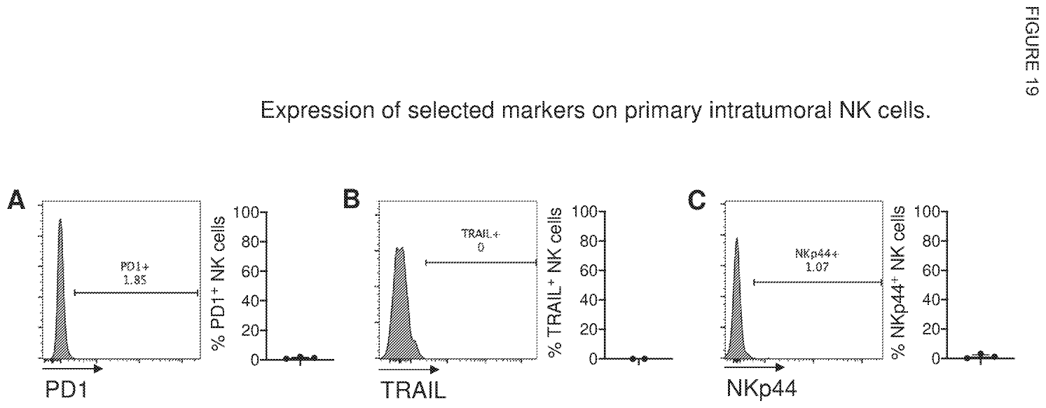

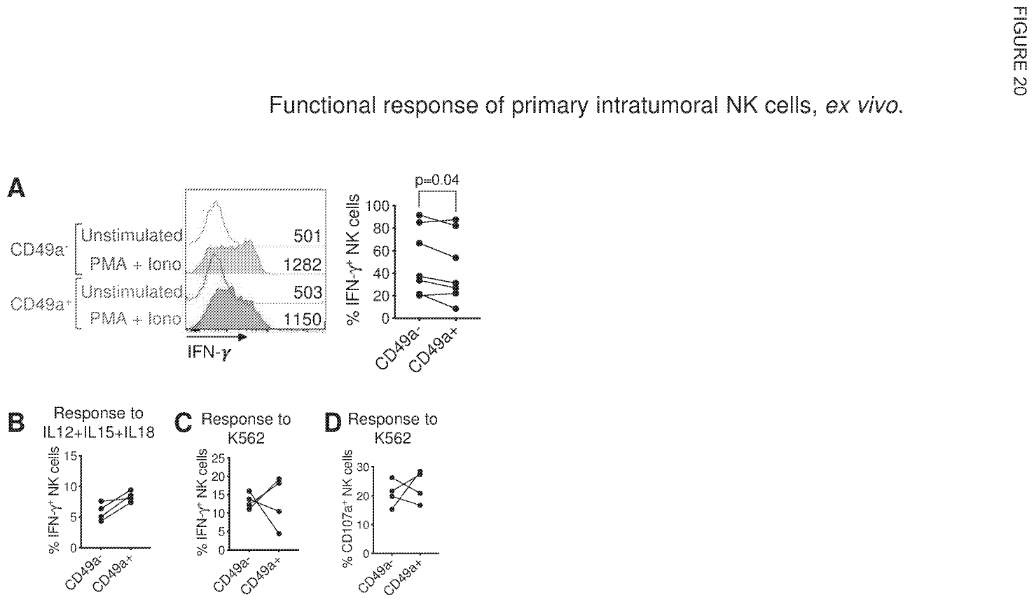

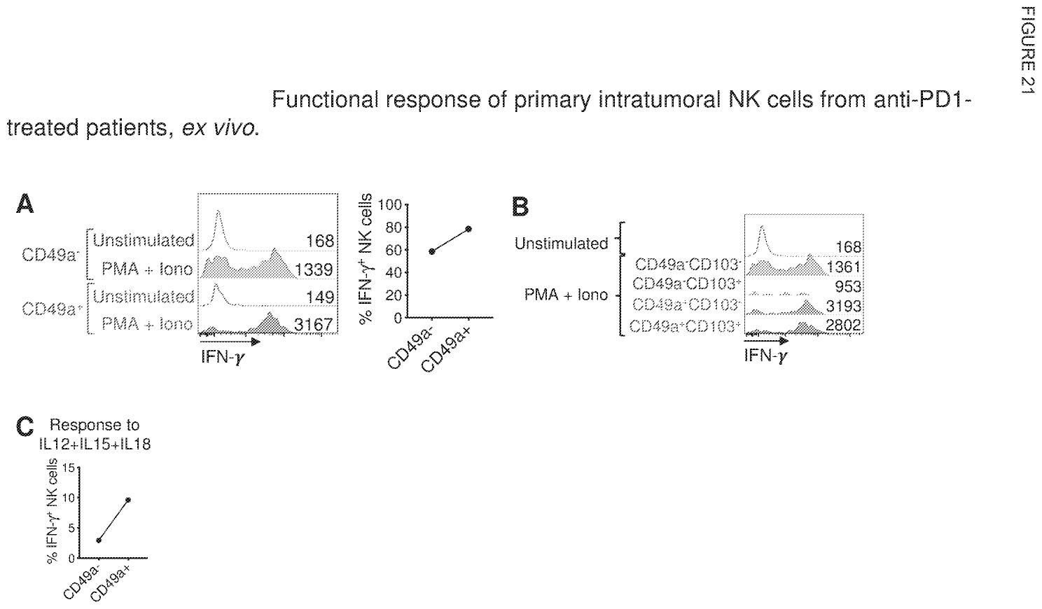

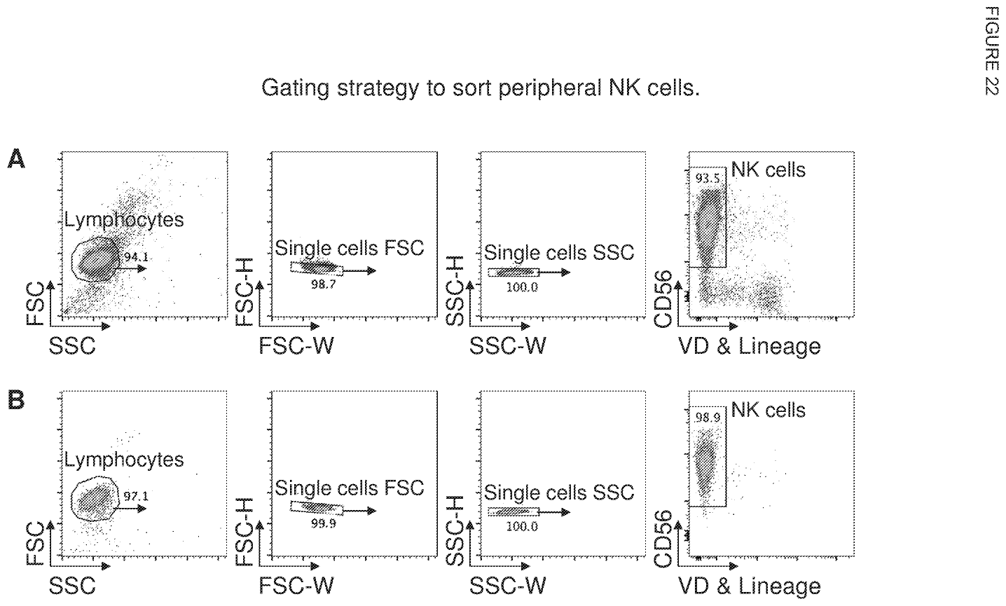

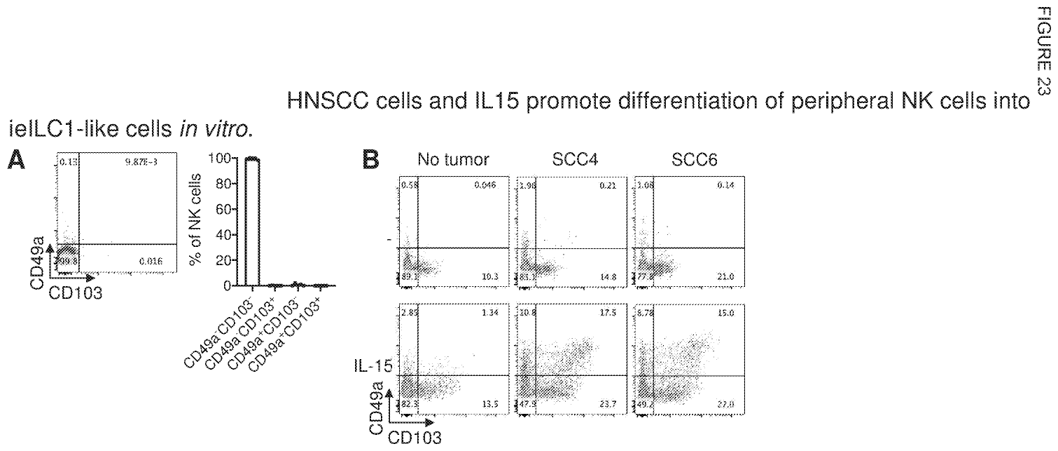

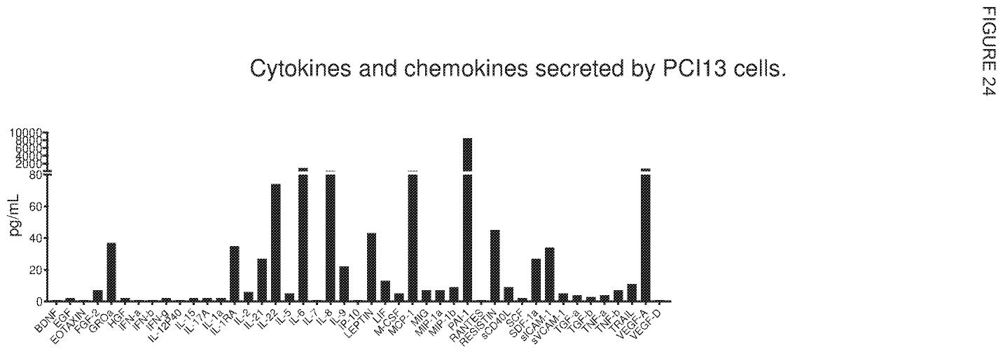

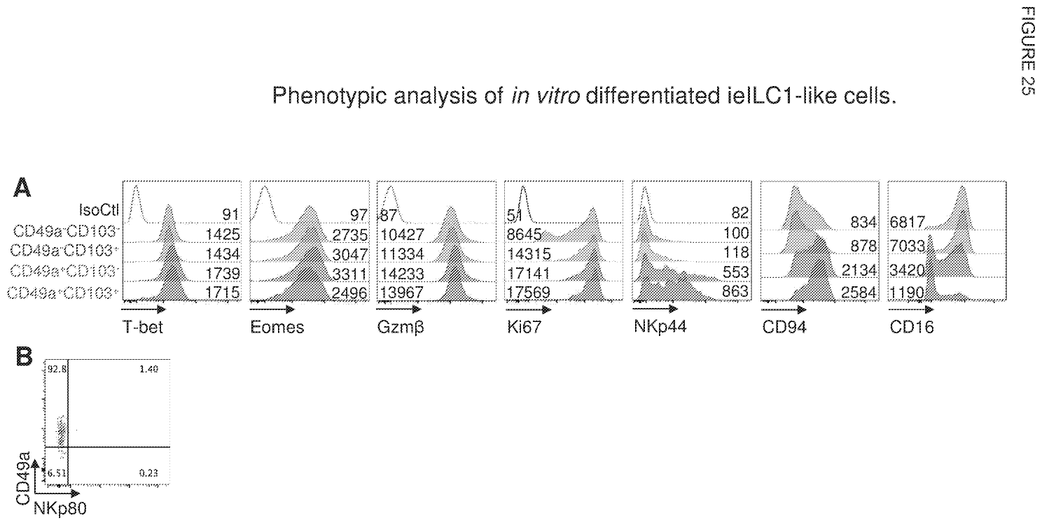

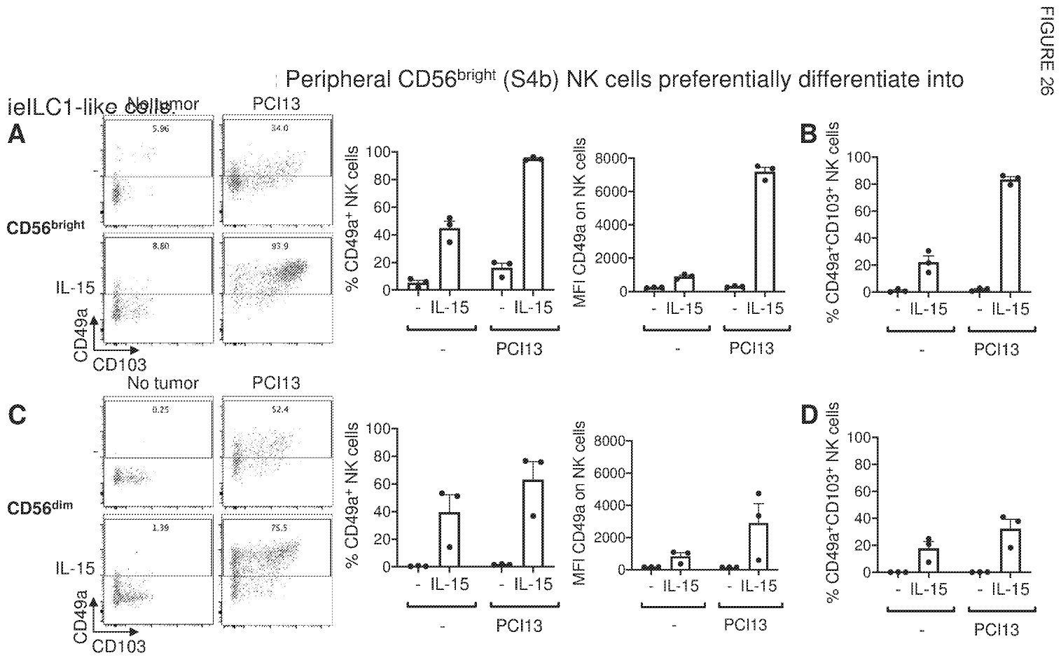

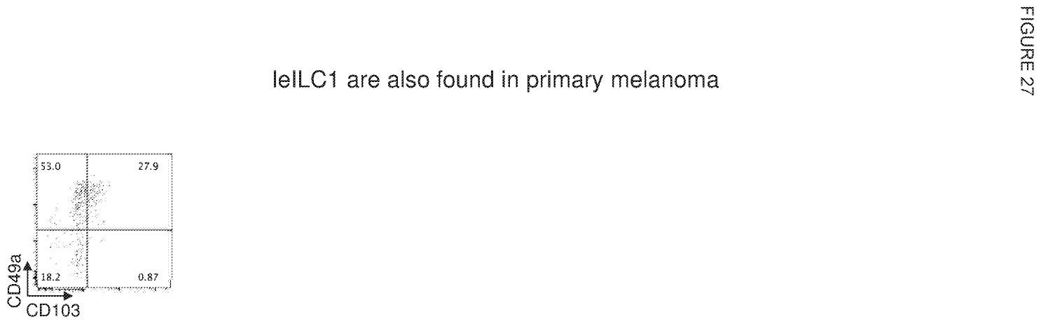

Further, as shown in FIG. 6, IL-15 promotes differentiation of peripheral NK into ielLC1-like cells in vivo. 10⁶ PCI13 tumor cells were subcutaneously injected in the flank of NSG mice, after 10-15 days (when the tumor were palpable) peripheral NK cells were obtained from blood and were injected intra-tumor. Mice were treated or not with IL15 (5 μg/mouse) every 2 days. After 6 days, tumors were harvested and the phenotype and function of intra-tumoral NK cells were assessed. The data indicate an increased level of ielLC1-like cells following treatment.

### Materials and Methods.

Here, we describe a method for in vitro differentiation of human peripheral NK cells into intra-epithelial innate lymphoid cells 1 (ielLC1)-like cells, which display significant anti-tumor effector functions.

### MATERIALS

50mL tubes (E&K scientific; Cat #EK-12270)
Antibodies: anti-CD3-APC-Cy7 (Clone: UCHT1; BioLegend; Cat #300426), anti-CD5-APC-Cy7 (Clone: L17F12; BioLegend; Cat #364010), anti-CD14-APC-Cy7 (Clone: HCD14; BioLegend; Cat #325620), anti-CD19-APC-Cy7 (Clone: HIB19; BioLegend; Cat #302218), anti-CD20-APC-Cy7 (Clone: 2H7; BioLegend; Cat #302314) and anti-CD56-BUV737 (Clone: NCAM16.2; BD Biosciences; Cat #564447)
Blood: leukoreduction system (LRS) chambers were obtained from the Stanford Blood Center.
Centrifuge.
FACS buffer: PBS (Mediatech; Cat #21-040-CV) containing 2% heat-inactivated Fetal Bovine Serum (Omega Scientific; Cat #FB-21), 1mM EDTA (Invitrogen; Cat #15575-038) and 1% Pen Strep (Gibco; Cat #15140-122).
FACS sorter. Different FACS sorter have been used: BD FACSAria Fusion, BD FACSAria IIU or BD FACSAria II instruments (BD Biosciences).
FicoII-Paque PREMIUM density gradient media (GE Healthcare; Cat #17544202).
Fixable Viability Dye eFluor™ 780 (eBioscience; Cat #65-0865-18).
HNSCC cells. We used several HNSCC cells, including PCI13, SCC4 and SCC6.
IL-15 (BioLegend; Cat #570306).
Media for co-culture: RPMI 1640 (Corning; Cat #10-040-CV) supplemented with 10% heat-inactivated Fetal Bovine Serum (Omega Scientific; Cat #FB-21), 1% Pen Strep (Gibco; Cat #15140-122), 55 μM 2-Mercaptoethanol (Gibco; Cat #21985-023), 1× MEM Non-Essential Amino-Acids (Gibco; Cat #11140-050), 1 mM Sodium Pyruvate (Gibco; Cat #11360-070), 10 mM HEPES (Gibco; Cat #25-060-CI).
Media for HNSCC cell culture: DMEM/F-12 50/50 (Corning; Cat #10-092-CV) supplemented with 10% heat-inactivated Fetal Bovine Serum (Omega Scientific; Cat #FB-21), 1% Pen Strep (Gibco; Cat #15140-122).
PBS (Mediatech; Cat #21-040-CV).
Plates. Different types of plates can be used depending on the amount of cells to be cultured; 5.10⁵ HNSCC cells per well on a 6-well plate (E&K scientific; Cat #EK-27160), 2.10⁵ HNSCC cells per well on a 12-well plate (E&K scientific; Cat #EK-45180), 10⁵ HNSCC cells per well on a 24-well plate (E&K scientific; Cat #EK-42160) or 10⁴ HNSCC cells per well on a 96-well plate (VWR International; Cat #82050-771).
RosetteSep™ Human NK Cell Enrichment Cocktail (Stem Cell Technologies; Cat #15065).

### Methods

### Seeding HNSCC Cells.

HNSCC cells were harvested from their culture, washed with PBS and counted.
HNSCC cells were then resuspended in media for co-culture, and seeded into plates as follows:

| | Amount of HNSCC | Volume for |
|---|---|---|
| Plate | cells | seeding |
| type | (per well) | (in mL) |
| 96-well | 10⁴ | 0.1 |
| 24-well | 10⁵ | 0.5 |
| 12-well | 2.10⁵ | 1 |
| 6-well | 5.10⁵ | 2 |

Note: Both, irradiated and live HNSCC were able to differentiate cNK cells into ielLC1-like cells.

### Blood Processing;

Blood was transferred into a 50mL tube and added PBS to a final volume of 15mL.
30 μL of RosetteSep™ Human NK Cell Enrichment Cocktail was added, and vortexed.
Blood was incubated 20 min at room temperature (RT), with intermittent vortexing.
On a separated 50 mL tube, 15 mL of Ficoll-Paque PREMIUM density gradient media were added.
Blood (from c) was carefully layered on top of the Ficoll-Paque PREMIUM density gradient media (from d) without mixing.
The tube was centrifuged at 300 g without break, during 20 min at RT.
The enriched NK cells (layer containing PBMCs) were recovered and transferred into a new 50 mL tube.
Enriched NK cells were washed 2 times with PBS.
Enriched NK cells were counted.

### Staining of Enriched NK Cells.

Enriched NK cells were resuspended with 2 mL of working solution of Fixable Viability Dye eFluor™ 780.
Enriched NK cells were washed with FACS buffer.
Enriched NK cells were resuspended with FACS buffer, 1 mL FACS buffer per 10⁷ enriched NK cells.
Antibody cocktail, containing at least the following antibodies, was added:

| | | Amount | | | |
|---|---|---|---|---|---|
| | | (for 10⁴ | | Catalog | |
| Specificity | Fluorophore | cells) | Clone | # | Company |
| CD3 | APC-Cy7 | 5 μL | UCHT1 | 300426 | BioLegend |
| CD5 | APC-Cy7 | 5 μL | L17F12 | 364010 | BioLegend |
| CD14 | APC-Cy7 | 5 μL | HCD14 | 325620 | BioLegend |
| CD19 | APC-Cy7 | 5 μL | HIB19 | 302218 | BioLegend |
| CD20 | APC-Cy7 | 5 μL | 2H7 | 302314 | BioLegend |
| CD56 | BUV737 | 5 μL | NCAM16.2 | 564447 | BD |
| | | | | | Biosciences |

Enriched NK cells were incubated during 30 min, at 4° C. in the dark.
Enriched NK cells were washed with FACS buffer.
Enriched NK cells were resuspended in media.

### FACS Sorting of NK Cells.

NK cells were defined as Lineage^{neg}CD56^{pos} lymphocytes. NK cells were sorted according with the following gating strategy:
Note: Alternatively, subpopulations of NK cells, such as CD56^{bright} NK cells, were sorted and differentiated using this protocol.
After the sort, the purity of NK cells was determined.
NK cells were washed with media.

### Co-Culture.

NK were resuspended with media containing IL-15.
NK cells were added to the co-culture as follows:

| | Amount of NK | Amount of HNSCC | | | |
|---|---|---|---|---|---|
| Plate | cells | cells | Ratio | Final volume | Final IL-15 |
| type | (per well) | (per well) | (effector:target) | (in mL) | concentration |
| 96-well | 10⁵ | 10⁴ | 10:1 | 0.25 | 10 ng/mL |
| 24-well | 10⁶ | 10⁵ | 10:1 | 1 | 10 ng/mL |
| 12-well | 2.10⁶ | 2.10⁵ | 10:1 | 2 | 10 ng/mL |
| 6-well | 5.10⁶ | 5.10⁵ | 10:1 | 5 | 10 ng/mL |

On day 2, half of the supernatant was replaced with fresh media containing IL-15 (20 ng/mL).
   Harvesting and use of ielLC1-Like Cells.
On day 4, ILC1-like cells were harvested.
Note: The harvested cells were ielLC1-like cells. HNSCC cells were killed during the culture.
ILC1-like cells were washed with PBS and counted.
ILC1-like cells were then used for phenotypic analysis and functional assays.

1. A method for differentiating mammalian natural killer cells to adapt an intraepithelial innate lymphoid cells (ielLC1)-like phenotype, the method comprising:
differentiating peripheral natural killer (NK) cells in the presence of IL-15 and epithelial cells or plate coatings that mimic features of epithelial cells, to generate CD49a+ CD103+ cells having features and phenotype of ielLC1s, with enhanced cytotoxic activity and expression of Th1 type cytokines.

2. The method of claim 1, wherein following a period of culture of from about 1 to about 7 days, the cells thus differentiated are isolated by selecting for ielLC1 markers.

3. The method of claim 1, comprising a step of selecting for cells that are positive for one or both of CD49a and CD103.

4. The method of claim 1, wherein the NK cells are human cells, optionally differentiated ex vivo.

5. (canceled)

6. The method of claim 4, wherein ex vivo differentiated cells are administered to a patient in an adoptive transfer for treatment of cancer.

7. The method of claim 1, wherein the NK are differentiated in vivo.

8. The method of claim 1, wherein the differentiating step comprises contacting the population of peripheral NK cells with culture medium comprising a dose of IL-15 and epithelial cells effective to differentiate the peripheral NK cells to CD49a+ CD103+ cells, optionally comprising an effective dose of TGF-β.

9. (canceled)

10. The method of claim 1, wherein the NK cells are purified from a human blood sample.

11. The method of claim 1, wherein the epithelial cells are carcinoma cells, optionally head and neck squamous cell carcinoma cells (HNSCC), and wherein the ratio of NK cells to epithelial cells is from about 100:1 to about 1:100.

12-16. (canceled)

17. A therapeutically effective dose of isolated, differentiated ielLC1-like cells produced by the method according to claim 1.

18. A method of treating a condition that benefits from enhanced cytotoxic activity and expression of Th1 type cytokines, the method comprising administering a dose of differentiated ielLC1-like of claim 17.

19. The method of claim 18, wherein the condition is cancer, selected from a hematologic cancer and a solid tumor.

20-21. (canceled)

22. The method of claim 19, wherein the solid tumor is a carcinoma.

23. The method of claim 19, wherein administration is systemic or intra-tumoral.

24. (canceled)

25. The method of claim 18, wherein administration is combined with additional anti-cancer therapies, optionally checkpoint inhibitors, tumor-specific antibodies, chemotherapy, radiation.

26. The method of claim 1, wherein the plate coatings that mimic features of epithelial cells are a non-cellular matrix, optionally plastic tissue culture plates coated with E-Cadherin in the absence of feeder layer cells.

27. (canceled)

28. The method of claim 1, wherein the peripheral NK cells are cryopreserved prior to the differentiating step.

29. The method of claim 1, wherein NKp44 is activated in the differentiating step.

30. (canceled)

31. The method of claim 1, wherein the differentiated ielLC1-like cells are genetically modified to generate chimeric antigen receptor (CAR) NK cells.
